(19) United States
(12) Patent Application Publication
(10) Pub. No.: US 2025/0144095 A1
(43) Pub. Date: May 8, 2025

(54) COMPOSITIONS AND METHODS FOR TREATMENT OF CONNECTIVE TISSUE DISORDERS

(71) Applicant: The Johns Hopkins University, Baltimore, MD (US)

(72) Inventors: Harry C. Dietz, Townson, MD (US); Caitlin J. Bowen, Baltimore, MD (US)

(21) Appl. No.: 18/835,516

(22) PCT Filed: Feb. 3, 2023
(86) PCT No.: PCT/US23/61972

§ 371 Date: Aug. 2, 2024

Related U.S. Application Data
(60) Provisional application No. 63/306,481, filed on Feb. 3, 2022.
(60) Provisional application No. 63/322,887, filed on Mar. 23, 2022.

(51) Int. Cl. A61K 31/506; A61K 31/407; A61K 31/4184; A61K 31/422; A61K 31/4523; A61P 19/00

(52) U.S. Cl. CPC A61K 31/506; A61K 31/407; A61K 31/4184; A61K 31/422; A61K 31/4523; A61P 19/00

(57) ABSTRACT

The present disclosure relates to compositions and methods for treating vascular Ehlers Danlos Syndrome and associated connective tissue disorders.

### CROSS-REFERENCES TO RELATED APPLICATION

This application claims the benefit of priority of 1) U.S. Provisional Application No. 63/306,481 filed Feb. 3, 2022 and 2) U.S. Provisional Application No. 63/322,887 filed Mar. 23, 2022, both of which applications are incorporated herein by reference in their entireties and for all purposes.

### STATEMENT REGARDING FEDERALLY SPONSORED RESEARCH

This invention was made with government support under grant AR041135 awarded by the National Institutes of Health. The government has certain rights in the invention.

### FIELD

The present disclosure relates to compositions and methods of treatment of connective tissue disorders.

### BACKGROUND

Vascular Ehlers Danlos Syndrome (vEDS) is an inherited connective tissue disorder caused by heterozygous mutations in the COL3A1 gene, resulting in spontaneous vascular and/or organ rupture. Attenuation of PKC/ERK pathway activation affords protection from vascular rupture in mouse models of vascular Ehlers Danlos syndrome (vEDS). The mechanism by which this intracellular signaling cascade is activated secondary to mutations in COL3A1 remains unknown.

### SUMMARY

Provided herein are, inter alia, compositions, formulations and methods for inhibiting, treating, preventing, and/or reducing the symptoms of severity of connective tissue disorders. Agents that modulate cellular signaling events are provided.

Accordingly, in certain embodiments, a method of treating a connective tissue disorder, comprises administering a therapeutically effective amount of an agent which modulates endothelin receptor activation, expression or function, and/or inhibits endothelin-1 expression or binding to the endothelin type A and endothelin type B receptors (EDNRA/B), thereby treating the connective tissue disorder.

In certain embodiments, the connective tissue disorder is Ehlers-Danlos Syndrome (EDS). In certain embodiments, the Ehlers-Danlos Syndrome (EDS) is hypermobile EDS, classical EDS, kyphoscoliosis EDS, arthrochalasia EDS, dermatosparaxis EDS, brittle cornea syndrome, classical-like EDS, spondylodysplastic EDS, musculocontractual EDS, myopathic EDS, periodontal EDS, cardiac-valcular EDS, or vascular EDS (vEDS). In certain embodiments, the Ehlers-Danlos Syndrome (EDS) is vascular EDS.

In certain embodiments, the agent comprises an antibody or fragment thereof, a polypeptide, a small molecule, a nucleic acid molecule, or any combination thereof.

In certain preferred embodiments, the agent is a small molecule.

In certain preferred embodiments, the agent is a small molecule endothelin receptor antagonist. Thus, preferred small molecule agents include EDNR antagonists, such as selective ETA receptor antagonists which affect endothelin A receptors or endothelin B receptors, or dual antagonists, which affect both endothelin A and B receptors.

In certain embodiments, the agent comprises bosentan, or a pharmaceutically acceptable salt thereof. In certain embodiments, the effective amount of the bosentan or the pharmaceutically acceptable salt thereof is from about 0.001 mg/kg to 250 mg/kg body weight of a patient.

In certain embodiments, the agent comprises sitaxentan, ambrisentan, acitentan and/or tezosentan. In certain embodiments, an effective amount of the sitaxentan, ambrisentan, acitentan and/or tezosentan is from about 0.001 mg/kg to 250 mg/kg body weight of a patient.

In additional exemplary embodiments, endothelin receptor antagonists for use in the present methods and compositions include atrasentan, BQ-123, zibotentan, edonentan, and/or macitentan. In certain embodiments, an effective amount of atrasentan, BQ-123, zibotentan, edonentan, and/or macitentan is from about 0.001 mg/kg to 250 mg/kg body weight of a patient.

In certain embodiments, the method further comprises administering an agent that modulates the activity or expression of protein kinase C (PKC), mitogen-activated protein kinase (MEK) or the combination thereof. In certain embodiments, a modulator of PKC activity or function, comprises ruboxistaurin or pharmaceutically acceptable salts thereof. In certain embodiments, a modulator of MEK activity or function, comprises trametinib, binimetinib, selumetinib, cobimetinib or pharmaceutically acceptable salts thereof. In certain embodiments, the method further comprises administering ruboxistaurin, cobimetinib pharmaceutically acceptable salts thereof or the combination thereof. In certain embodiments, the method further comprises a modulator of type III collagen expression or function.

In certain embodiments, a method of treating a vascular Ehlers-Danlos Syndrome in a subject in need thereof is provided, the method comprising: administering to the subject an effective amount of bosentan or a pharmaceutically acceptable salt thereof. In certain embodiments, the method further comprises the effective amount of the bosentan or the pharmaceutically acceptable salt thereof is from about 0.001 mg/kg to 250 mg/kg body weight. In certain embodiments, the method further comprises administering an agent for modulating expression or activity of a COL3A1 gene, correcting mutations of a COL3A1 gene or the combination thereof.

In embodiments, the method comprises administering an effective amount of the agent. The effective amount of the agent is from about 0.001 mg/kg to about 250 mg/kg body weight, e.g., about 0.001 mg/kg, about 0.05 mg/kg, about 0.1 mg/kg, about 0.5 mg/kg, about 1 mg/kg, about 5 mg/kg, about 10 mg/kg, about 25 mg/kg, about 50 mg/kg, about 75 mg/kg, about 100 mg/kg, about 125 mg/kg, about 150 mg/kg, about 175 mg/kg, about 200 mg/kg, about 225 mg/kg, or about 250 mg/kg body weight. Ultimately, the attending physician or veterinarian decides the appropriate amount and dosage regimen.

In some cases, the agent is administered at least once per day, at least once per week, or at least once per month. The agent suitably may be administered for a duration of one day, one week, one month, two months, three months, six months, 9 months, or one year. In some cases, the agent is administered daily, e.g., every 24 hours. Or, the agent is administered continuously or several times per day, e.g., every 1 hour, every 2 hours, every 3 hours, every 4 hours, every 5 hours, every 6 hours, every 7 hours, every 8 hours, every 9 hours, every 10 hours, every 11 hours, or every 12 hours.

Furthermore, the methods described herein prevent or reduce the severity of a vasculopathy (vEDS) by at least about 1%, e.g., at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99%.

A variety of administration routes are available. For example, the agent is administered topically, orally, via inhalation, or via injection.

The subject is preferably a mammal in need of such treatment or prophylaxis, e.g., a subject that has been diagnosed with a vasculopathy or a predisposition thereto. The mammal is any mammal, e.g., a human, a primate, a mouse, a rat, a dog, a cat, a horse, as well as livestock or animals grown for food consumption, e.g., cattle, sheep, pigs, chickens, and goats. In a preferred embodiment, the mammal is a human.

In aspects, a subject who has or is at risk of suffering from a connective tissue disorder, e.g., a vasculopathy (and in certain embodiments vEDS), has a level of MEK or PKC protein or mRNA that is different than a normal control. In some embodiments, a test sample obtained from the subject comprises a level of MEK or PKC protein or mRNA that is different than a normal control. For example, the test sample may comprise a level of MEK or PKC protein or mRNA that is at least about 5%, 10%, 15%, 20%, 25%, 30%, 40%, 45%, 50%, 55%, 60%, 65%, 70%, 75%, 80%, 90%, 95%, 99%, 100%, 5-50%, 50-75%, 75-100%, 1-fold, 2-fold, 3-fold, 4-fold, or 5-fold higher compared to a normal control.

In certain embodiments, the agent decreases the PKC protein or mRNA level by at least about 5%, 10%, 15%, 20%, 25%, 30%, 40%, 45%, 50%, 55%, 60%, 65%, 70%, 75%, 80%, 90%, 95%, 99%, 100%, 5-50%, 50-75%, 75-100%, 1-fold, 2-fold, 3-fold, 4-fold, or 5-fold compared to a normal control. In other embodiments, the agent decreases the level of PKC activity by at least about 5%10%, 15%, 20%, 25%, 30%, 40%, 45%, 50%, 55%, 60%, 65%, 70%, 75%, 80%, 90%, 95%, 99%, 100%, 5-50%, 5-fold, compared to a normal control.

In some embodiments, the agent decreases the mitogen-activated protein kinase (MEK) protein or mRNA level by at least about 5%, 10%, 15%, 20%, 25%, 30%, 40%, 45%, 50%, 55%, 60%, 65%, 70%, 75%, 80%, 90%, 95%, 99%, 100%, 5-50%, 50-75%, 75-100%, 1-fold, 2-fold, 3-fold, 4-fold, or 5-fold compared to a normal control. In other embodiments, the agent decreases the level of MEK activity by at least about 5%, 10%, 15%, 20%, 25%, 30%, 40%, 45%, 50%, 55%, 60%, 65%, 70%, 75%, 80%, 90%, 95%, 99%, 100%, 5-50%, 5-fold, compared to a normal control.

In certain embodiments, a method of treating a vascular Ehlers-Danlos Syndrome further comprises administering a gene editing agent. In certain embodiments, the gene editing agent is a CRISPR-associated endonuclease is a Type I, Type II, or Type III Cas endonuclease. In certain embodiments, the CRISPR-associated endonuclease is a Cas9 endonuclease, a Cas12 endonuclease, a Cas 13 endonuclease, a CasX endonuclease, a CasΦ endonuclease or variants thereof. In certain embodiments, the CRISPR-associated endonuclease is a Cas9 nuclease or variants thereof. In certain embodiments, the Cas9 nuclease is a Staphylococcus aureus Cas9 nuclease. In certain embodiments, the Cas9 variant comprises one or more point mutations, relative to wildtype Streptococcus pyogenes Cas9 (spCas9), selected from the group consisting of: R780A, K810A, K848A, K855A, H982A, K1003A, R1060A, D1135E, N497A, R661A, Q695A, Q926A, L169A, Y450A, M495A, M694A, and M698A. In certain embodiments, a Cas9 variant comprises a human-optimized Cas9; a nickase mutant Cas9; saCas9; enhanced-fidelity SaCas9 (efSaCas9); SpCas9(K855a); SpCas9(K810A/K1003A/r1060A); SpCas9(K848A/K1003A/R1060A); SpCas9 N497A, R661A, Q695A, Q926A; SpCas9 N497A, R661A, Q695A, Q926A, D1135E; SpCas9 N497A, R661A, Q695A, Q926A L169A; SpCas9 N497A, R661A, Q695A, Q926A Y450A; SpCas9 N497A, R661A, Q695A, Q926A M495A; SpCas9 N497A, R661A, Q695A, Q926A M694A; SpCas9 N497A, R661A, Q695A, Q926A H698A; SpCas9 N497A, R661A, Q695A, Q926A, D1135E, L169A; SpCas9 N497A, R661A, Q695A, Q926A, D1135E, Y450A; SpCas9 N497A, R661A, Q695A, Q926A, D1135E, M495A; SpCas9 N497A, R661A, Q695A, Q926A, D1135E, M694A; SpCas9 N497A, R661A, Q695A, Q926A, D1135E, M698A; SpCas9 R661A, Q695A, Q926A; SpCas9 R661A, Q695A, Q926A, D1135E; SpCas9 R661A, Q695A, Q926A, L169A; SpCas9 R661A, Q695A, Q926A Y450A; SpCas9 R661A, Q695A, Q926A M495A; SpCas9 R661A, Q695A, Q926A M694A; SpCas9 R661A, Q695A, Q926A H698A; SpCas9 R661A, Q695A, Q926A D1135E L169A; SpCas9 R661A, Q695A, Q926A D1135E Y450A; SpCas9 R661A, Q695A, Q926A D1135E M495A; or SpCas9 R661A, Q695A, Q926A, D1135E or M694A. In certain embodiments, the CRISPR-associated endonuclease is optimized for expression in a human cell. In certain embodiments, the gene editing agent comprises one or more guide RNAs (gRNAs) complementary to a target sequence within the COL3A1 gene. In certain embodiments, the gene editing agent comprises two or more guide RNAs (gRNAs) complementary to a target sequence within the COL3A1 gene, wherein each nucleic acid target sequence in the COL3A1 gene is different. In certain embodiments, the target nucleic acid sequence is a COL3A1 gene regulatory sequence, e.g. a COL3A1 promoter sequence, a COL3A1 enhancer sequence. In certain embodiments, the gene editing agent suppresses COL3A1 expression or corrects the mutations of the COL3A1 gene. In certain embodiments, a nucleic acid sequence for administering to the subject comprises a corrected or wild type COL3A1 nucleic acid or COL3A1 fragments comprising the wild type sequences ((NCBI Accession No: NM_000090.3; HGNC: 2201; NCBI Entrez Gene: 1281; Ensembl: ENSG00000168542; OMIM®: 120180; UniProtKB/Swiss-Prot: P02461).

In certain embodiments, the isolated nucleic acid sequences are included in at least one expression vector selected from the group consisting of: a lentiviral vector, an adenovirus vector, an adeno-associated virus vector, a vesicular stomatitis virus (VSV) vector, a pox virus vector, and a retroviral vector. In certain embodiments, the expression vector comprises: a lentiviral vector, an adenoviral vector, or an adeno-associated virus vector. In certain embodiments, the adeno-associated virus (AAV) vector is AV1, AAV2, AAV3, AAV4, AAV5, AAV6, AAV7, AAV8, AAV9, AAV10, AAV11, AAVDJ, or AAVDJ/8. In certain embodiments, the vector comprising the nucleic acid further comprises a promoter. In certain embodiments, the promoter comprises a ubiquitous promoter, a tissue-specific promoter, an inducible promoter or a constitutive promoter.

In certain embodiments, detection of endothelin-1 (ET1) signaling and/or levels of ET1, nitric oxide, nitrate, or nitrite in patient sample are indicative of vascular disease risk, progression, and/or therapeutic response in the patient.

In certain embodiments, a composition comprises one or more biomarkers, wherein the biomarkers comprise: endothelin-1 (ET1), ET1 signalling, nitric oxide, nitrate, or nitrite. In certain embodiments, detection of ET1 signaling and/or levels of ET1, nitric oxide, nitrate, or nitrite in patient sample are indicative of vascular disease risk, progression, and/or therapeutic response in the patient.

Suitable endothelin receptor antagonist, including small molecule endothelin receptor agonists for use in the present methods and compostions are disclosed herein and can be readily identified, including by assays such as disclosed in U.S. Pat. No. 5,334,598 where the candidate endothelin receptor antagonist compound suitably exhibits an IC₅₀ or ED₅₀ of a desired threshold value such 10⁻³ or lower or 10⁻⁴ or lower in standard in vitro assays that assess endothelin receptor antagonist activity such as disclosed in U.S. Pat. No. 5,334,598.

Any compositions or methods provided herein can be combined with one or more of any of the other compositions and methods provided herein.

### Definitions

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the present disclosure, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods. In specific instances, unless otherwise indicated, “about” is ±10%.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

As used herein, the term “agent” is meant to encompass any molecule, chemical entity, composition, drug, therapeutic agent, chemotherapeutic agent, or biological agent capable of preventing, ameliorating, or treating a disease or other medical condition. The term includes small molecule compounds, antisense reagents, siRNA reagents, gene editing agents (e.g. CRISPR/Cas) antibodies, enzymes, peptides organic or inorganic molecules, natural or synthetic compounds and the like. An agent can be assayed in accordance with the methods of the disclosure at any stage during clinical trials, during pre-trial testing, or following FDA-approval.

In the descriptions herein and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements—or, as appropriate, equivalents thereof—and that other elements can be included and still fall within the scope/definition of the defined item, composition, apparatus, method, process, system, etc.

A “control” sample or value refers to a sample that serves as a reference, usually a known reference, for comparison to a test sample. For example, a test sample can be taken from a test subject, e.g., a subject with a connective tissue disorder such as a vasculopathy (e.g., vEDS) or in need of diagnosis, and compared to samples from known conditions, e.g., a subject (or subjects) that does not have the disease (a negative or normal control), or a subject (or subjects) who does have the disease (positive control). A control can also represent an average value gathered from a number of tests or results. One of skill in the art will recognize that controls can be designed for assessment of any number of parameters. One of skill in the art will understand which controls are valuable in a given situation and be able to analyze data based on comparisons to control values. Controls are also valuable for determining the significance of data. For example, if values for a given parameter are variable in controls, variation in test samples will not be considered as significant.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a symptom of the disease or disorder, the frequency with which such a symptom is experienced by a patient, or both, is reduced.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

An “effective amount” or “therapeutically effective amount” of a compound is that amount of compound which is sufficient to provide a beneficial effect to the subject to which the compound is administered. An “effective amount” of a delivery vehicle is that amount sufficient to effectively bind or deliver a compound.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

The word “expression” or “expressed” as used herein in reference to a nucleic acid sequence (e.g. a gene) means the transcriptional and/or translational product of that sequence. The level of expression of a DNA molecule in a cell may be determined on the basis of either the amount of corresponding mRNA that is present within the cell or the amount of protein encoded by that DNA produced by the cell (Sambrook et al., 1989 Molecular Cloning: A Laboratory Manual, 18.7-18.88). When used in reference to polypeptides, expression includes any step involved in the production of a polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc).

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared ×100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native Environment such as, for example, a host cell. In the context of the present disclosure, the following abbreviations for the commonly occurring nucleic acid bases are used. “A” refers to adenosine, “C” refers to cytosine, “G” refers to guanosine, “T” refers to thymidine, and “U” refers to uridine.

By the term “modulate,” it is meant that any of the mentioned activities, are, e.g., increased, enhanced, increased, agonized (acts as an agonist); or, decreased, reduced, suppressed blocked, or antagonized (acts as an antagonist). Modulation can increase activity more than 1-fold, 2-fold, 3-fold, 5-fold, 10-fold, 100-fold, etc., over baseline values. Modulation can also decrease its activity below baseline values. Modulation can also normalize an activity to a baseline value.

The term, “normal amount” with respect to a compound (e.g., a protein or mRNA) refers to a normal amount of the compound in an individual who does not have a connective tissue disorder such as a vasculopathy (e.g., vEDS) or in a healthy or general population. The amount of a compound can be measured in a test sample and compared to the “normal control” level, utilizing techniques such as reference limits, discrimination limits, or risk defining thresholds to define cutoff points and abnormal values (e.g., for a particular vEDS or a symptom thereof). The normal control level means the level of one or more compounds or combined compounds typically found in a subject known not suffering from a vEDS. Such normal control levels and cutoff points may vary based on whether a compounds is used alone or in a formula combining with other compounds into an index. Alternatively, the normal control level can be a database of compounds patterns from previously tested subjects who did not develop a vEDS or a particular symptom thereof (e.g., in the event the vEDS develops or a subject already having the vEDS is tested) over a clinically relevant time horizon.

The level that is determined may be the same as a control level or a cut off level or a threshold level, or may be increased or decreased relative to a control level or a cut off level or a threshold level. In some aspects, the control subject is a matched control of the same species, gender, ethnicity, age group, smoking status, body mass index (BMI), current therapeutic regimen status, medical history, or a combination thereof, but differs from the subject being diagnosed in that the control does not suffer from the disease (or a symptom thereof) in question or is not at risk for the disease.

Relative to a control level, the level that is determined may an increased level. As used herein, the term “increased” with respect to level (e.g., protein or mRNA level) refers to any % increase above a control level. In various embodiments, the increased level may be at least or about a 5% increase, at least or about a 10% increase, at least or about a 15% increase, at least or about a 20% increase, at least or about a 25% increase, at least or about a 30% increase, at least or about a 35% increase, at least or about a 40% increase, at least or about a 45% increase, at least or about a 50% increase, at least or about a 55% increase, at least or about a 60% increase, at least or about a 65% increase, at least or about a 70% increase, at least or about a 75% increase, at least or about a 80% increase, at least or about a 85% increase, at least or about a 90% increase, at least or about a 95% increase, relative to a control level

Relative to a control level, the level that is determined may a decreased level. As used herein, the term “decreased” with respect to level (e.g., protein or mRNA level) refers to any % decrease below a control level. In various embodiments, the decreased level may be at least or about a 5% decrease, at least or about a 10% decrease, at least or about a 15% decrease, at least or about a 20% decrease, at least or about a 25% decrease, at least or about a 30% decrease, at least or about a 35% decrease, at least or about a 40% decrease, at least or about a 45% decrease, at least or about a 50% decrease, at least or about a 55% decrease, at least or about a 60% decrease, at least or about a 65% decrease, at least or about a 70% decrease, at least or about a 75% decrease, at least or about a 80% decrease, at least or about a 85% decrease, at least or about a 90% decrease, at least or about a 95% decrease, relative to a control level.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

“Parenteral” administration of a composition includes, e.g., subcutaneous (s.c.), intravenous (i.v.), intramuscular (i.m.), or intrasternal injection, or infusion techniques.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The terms “pharmaceutically acceptable” (or “pharmacologically acceptable”) refer to molecular entities and compositions that do not produce an adverse, allergic or other untoward reaction when administered to an animal or a human, as appropriate. The term “pharmaceutically acceptable carrier,” as used herein, includes any and all solvents, dispersion media, coatings, antibacterial, isotonic and absorption delaying agents, buffers, excipients, binders, lubricants, gels, surfactants and the like, that may be used as media for a pharmaceutically acceptable substance.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

As used in this specification and the appended claims, the term “or” is generally employed in its sense including “and/or” unless the content clearly dictates otherwise.

A “tissue-specific” promoter is a nucleotide sequence which, when operably linked with a polynucleotide encodes or specified by a gene, causes the gene product to be produced in a cell substantially only if the cell is a cell of the tissue type corresponding to the promoter.

The term “target nucleic acid” or “target sequence” refer to a nucleic acid (often derived from a biological sample), to which the oligonucleotide is designed to specifically hybridize. It is either the presence or absence of the target nucleic acid that is to be detected, or the amount of the target nucleic acid that is to be quantified. The target nucleic acid has a sequence that is complementary to the nucleic acid sequence of the corresponding oligonucleotide directed to the target. The term target nucleic acid may refer to the specific subsequence of a larger nucleic acid to which the oligonucleotide is directed or to the overall sequence (e.g., gene or mRNA) whose expression level it is desired to detect. The difference in usage will be apparent from context.

A “therapeutic” treatment is a treatment administered to a subject who exhibits signs of pathology, for the purpose of diminishing or eliminating those signs.

As used herein, “treating a disease or disorder” means reducing the frequency with which a symptom of the disease or disorder is experienced by a patient. Disease and disorder are used interchangeably herein.

The phrase “therapeutically effective amount,” as used herein, refers to an amount that is sufficient or effective to prevent or treat (delay or prevent the onset of, prevent the progression of, inhibit, decrease or reverse) a disease or condition, including alleviating symptoms of such diseases.

To “treat” a disease as the term is used herein, means to reduce the frequency or severity of at least one sign or symptom of a disease or disorder experienced by a subject.

“Variant” as the term is used herein, is a nucleic acid sequence or a peptide sequence that differs in sequence from a reference nucleic acid sequence or peptide sequence respectively, but retains essential properties of the reference molecule. Changes in the sequence of a nucleic acid variant may not alter the amino acid sequence of a peptide encoded by the reference nucleic acid, or may result in amino acid substitutions, additions, deletions, fusions and truncations. Changes in the sequence of peptide variants are typically limited or conservative, so that the sequences of the reference peptide and the variant are closely similar overall and, in many regions, identical. A variant and reference peptide can differ in amino acid sequence by one or more substitutions, additions, deletions in any combination. A variant of a nucleic acid or peptide can be a naturally occurring such as an allelic variant, or can be a variant that is not known to occur naturally. Non-naturally occurring variants of nucleic acids and peptides may be made by mutagenesis techniques or by direct synthesis.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

Ranges: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

As used herein, “vasculitis (angiitis or angitis)” refers to inflammation of a blood vessel, e.g., arteritis, phlebitis, or lymphatic vessel, e.g., lymphangitis. Vasculitis can take various forms such as cutaneous vasculitis, urticarial vasculitis, leukocytoclastic vasculitis, livedo vasculitis and nodular vasculitis. Small vessel vasculitis may refer to inflammation of small or medium sized blood or lymphatic vessel, e.g., capillaries, venules, arterioles and arteries.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 is a series of plots showing the RNAseq read analysis of Edn1, Ednra, and Ednrb in the proximal descending aorta comparing wild type (n=3) and Col3a1^{G938D/+} aortas (n=3). Error bars show mean±s.e. P-values reported calculated using Student's t-test.

FIG. 2 is a series of scans of photographs demonstrating that endothelin-1 expression is enriched in in the descending thoracic aorta media of Col3a1^{G938D/+} mice. Representative immunofluorescence images showing levels of p-ERK1/2 (green) and Endothelin-1 (red) in the descending thoracic aorta of 8-week-old control (Col3a1^{+/+}) and mutant (Col3a1^{G938D/+}) mice. Scale bars: 50 m. Experiment was conducted at least 3 times.

FIG. 3 shows a representative Western blot analysis of Endothelin-1 comparing Col3a1^{+/+} (n=4) to Col3a1^{G938D/+} (n=5) proximal descending aortas and quantification of Endothelin-1 levels normalized to β-actin loading control for vEDS aortas. Error bars show mean±s.e. Asterisks signify significant differences using Student's t-test (*p<0.05).

FIG. 4 shows a representative Western blot analysis of Endothelin-1 comparing Col3a1^{+/+} (n=4) to Col3a1^{G938D/+} (n=5) proximal descending aortas from the C57BL/6J and 129Sve background and quantification of Endothelin-1 levels normalized to β-actin loading control for vEDS aortas. Error bars show mean±s.e. Asterisks signify significant differences using two-way ANOVA with Sidak's multiple comparisons post-hoc test (***p<0.001 (C57BL/6J Col3a1^{+/+} vs Col3a1^{G938D/+})).

FIG. 5 is a plot demonstrating an RNAseq read analysis of Ece1 in the proximal descending aorta comparing wild type (n=3) and Col3a1^{G938D/+} aortas (n=3). Error bars show mean±s.e. P-values reported calculated using Student's t-test.

FIG. 6 shows a Kaplan-Meier survival curve comparing Col3a1^{G938D/+} (n=93) to Col3a1^{G938D/+} (n=30) mice receiving bosentan. For all survival curves, significant differences were calculated using Log-Rank (Mantel-Cox) analysis and a universal control group with N=93 across all drug tests that started at P21.

FIG. 7A is a representative Western blot analysis of pPKCβ and pERK comparing Col3a1^{+/+} to Col3a1^{G938D/+} proximal descending aortas. FIG. 7B: is a plot showing the quantification of pPKCβ levels normalized to f-actin loading control comparing Col3a1^{+/+} (n=3) to Col3a1^{G938D/+} (n=3) and Col3a1^{G938D/+} Bosentan (n=5) aortas. Error bars show mean±s.e. FIG. 7C: is a plot showing the quantification of pERK levels normalized to f-actin loading control comparing Col3a1^{+/+} (n=3) to Col3a1^{G938D/+} (n=3) and Col3a1^{G938D/+} Bosentan (n=5) aortas. Error bars show mean±s.e. Asterisks signify significant differences using one-way ANOVA with Dunnett's multiple comparisons post-hoc test (**p<0.01, DF=2, F=11.19 (pPKC), DF=2, F=3.102 (pERK)).

FIG. 8 is a Kaplan-Meier survival curve comparing Col3a1^{G938D/+} Cre negative mice (ET1^{+/+}, ET1^{fl/+}, or ET1^{fl/fl}) (n=25) to Tie2Cre-ET1^{fl/+}-Col3a1^{G938D/+} (n=15) and Sm22Cre-ET1^{fl/+}-Col3a1^{G938D/+} mice. For all survival curves, significant differences were calculated using Log-Rank (Mantel-Cox) analysis (*p<0.05).

FIG. 9 is a series of scans of photographs showing that endothelin-1 expression is enriched in in the descending thoracic aorta media of Col3a1^{G938D/+} mice but reduced upon treatment with cobimetinib (MEKi) or ruboxistaurin (PKCi). Representative immunofluorescence images showing levels of p-ERK1/2 (green) and Endothelin-1 (red) in the descending thoracic aorta of 8-week-old control (Col3a1^{+/+}) and mutant (Col3a1^{G938D/+}) mice treated with cobimetinib and ruboxistaurin. Scale bars: 50 m. Experiment was conducted at least 3 times.

FIG. 10 is a plot showing the endothelin-1 serum levels comparing wild type (n=9) and Col3a1^{G938D/+} aortas (n=9). Error bars show mean±s.e. P-value calculated using Student's t-test. P=0.2448.

FIG. 11 is a plot showing the endothelin-1 urine levels comparing wild type (n=2) and Col3a1^{G938D/+} aortas (n=2). Error bars show mean±s.e. P-value calculated using Student's t-test. P=0.7284.

FIG. 12 is a plot showing the nitrate serum levels comparing wild type (n=5) and Col3a1^{G938D/+} aortas (n=5). Error bars show mean±s.e. P-value calculated using Student's t-test. P=0.6311.

### DETAILED DESCRIPTION

A role for Endothelin-1 in vascular rupture risk in vEDS mice is identified. Compositions and methods of treatment are provided.

### Connective Tissue Disorders

Connective tissue disease refers to a group of disorders involving the protein-rich tissue that supports organs and other parts of the body. Examples of connective tissue are fat, bone, and cartilage. These disorders often involve the joints, muscles, and skin, but they can also involve other organs and organ systems, including the eyes, heart, lungs, kidneys, gastrointestinal tract, and blood vessels. There are more than 200 disorders that affect the connective tissue. Causes and specific symptoms vary by the different types.

Examples of tissue diseases (e.g. epithelial, connective, muscle and nervous tissue) potentially treatable with the compositions and methods described herein include, but are not limited to the following: autoimmune, degenerative, inflammatory, infectious, cancerous, viral, fungal, injured or trauma derived. These tissue and/or organ diseases may be the primary disease or may be caused by an existing disease and/or illness. Examples include amyloidosis, atiral fibrillation, convulsion, cramp, dermatomyositis, enchondroma, fibroma, lumbao, heritable connective tissue disorder (e.g., Marfan syndrome, Peyronie's disease, Ehlers-Danlos syndrome, Osteogenesis imperfecta, Stickler syndrome, Alport syndrome, Congenital contractural arachnodactyly), autoimmune connective tissue disorder (e.g., systemic lupus erythematosus (SLE), rheumatoid arthritis, Scleroderma, Sjoegren's syndrome, mixed connective tissue disease, psoriatic arthritis), scurvy, muscle disease (e.g., muscle tumour, muscular dystrophy, disuse atrophy, denervation atrophy, Duchenne muscular dystrophy, facioscapulohumoral muscular dystrophy), hepatic disease, myasthenia gravis, myopathy, myositis, myositis ossificans, cancer, fibromyalgia, muscle fatigue, spasm, spasticity, sprain, strain, brain injury, spinal cord injury, gliomas, neuroeptheliomatous, hypertension, cardiovascular disease, diabetes, Alzheimer's disease, cystitis, AIDS, rickets, and nerve sheath tumors. Examples of tissues, organs and/or body systems affected by disease and may be treated with the compositions, and methods described therein, but are not limited to the following: Immune system, senory organs (e.g., organs of tase, smell, sight, hearing), digestive system (e.g., mouth, fauces, pharynx, esophagus, abdomen, stomach, small intestine, large intestine, liver, pancreas), urogenital apparatus, endocrinological systemt, metabolism, cardiovascular system (e.g., heart, blood pressure, arteries), hematology (e.g., blood chemistry), urinary organs (e.g., kidneys, ureters, urinary bladder, male urethra, female urethra, male gential organs (e.g., testes and their covering, ductus deferens, vesiculae seminales, ejaculatory ducts, penis, prostate, bulbourethral glands), female genital organs (e.g., ovaries, uterine tube, uterus, vagina, clitoris, Bartholin's glands, external organs, mammae)), ductless glands (e.g., thyroid, parathyroid, thymus, hypophysis cerebri, pineal body, chromaphil and corticol systems, spleen), reproduction, respiratory (e.g., larynx, trachea, bonchi, pleurae, mediastinum, lungs), central nervous system (e.g., nerves, nerve fibers), skin, epithelial (e.g., simple, stratified, pseudostratified columnar, glandular), connective (e.g., loose connective (e.g., areolar, adipose, reticular), and dense connective (e.g., dense regular, dense irregular)), cartilage (e.g., Hyaline, elastic, fibrous), muscle (e.g., skeletal muscle (e.g., type I, II, IIa, IIx, IIb), cardiac muscle, smooth muscle), nervous (e.g., neuron (e.g., motor neurons, interneuron, sensory neuron), neuroglia, spinal cord, nerves, brain).

In embodiments, the connective tissue disorder comprises a vasculopathy (e.g., vascular Ehlers-Danlos Syndrome), Marfan Syndrome, Loeys-Dietz Syndrome, or Familal thoracic aortic aneurysm.

### Ehlers-Danlos Syndromes (EDSs)

Ehlers-Danlos syndromes (EDSs) are a group of genetic connective tissue disorders. Symptoms may include loose joints, stretchy skin, and abnormal scar formation. These can be noticed at birth or in early childhood. Complications may include aortic dissection, joint dislocations, scoliosis, chronic pain, or early osteoarthritis.

EDSs are due to a mutation in one of more than a dozen different genes. The specific gene affected determines the specific EDS. Some cases result from a new mutation occurring during early development, while others are inherited in an autosomal dominant or recessive manner. This results in defects in the structure or processing of collagen. The diagnosis may be confirmed with genetic testing or a skin biopsy. People may be misdiagnosed with hypochondriasis, depression, or chronic fatigue syndrome.

To date, no cure is known, however, physical therapy and bracing may help strengthen muscles and support joints. While some disorders result in a normal life expectancy, those that affect blood vessels generally result in a shorter life expectancy. EDSs affect about one in 5,000 people globally, and the prognosis depends on the specific disorder.

### EDS Classification

Hypermobile EDS (type 3 hEDS) is characterized primarily by joint hypermobility affecting both large and small joints, which may lead to recurrent joint dislocations and subluxations (partial dislocation). In general, people with this type have soft, smooth, and velvety skin with easy bruising and chronic pain of the muscles and/or bones. The mutation that causes this type of EDS is unknown. Less skin involvement is seen than other types. No genetic test for this type is available.

Classical EDS (type 1 cEDS) is associated with extremely elastic (stretchy), smooth skin that is fragile and bruises easily; wide, atrophic scars (flat or depressed scars); and joint hypermobility. Molluscoid pseudotumors (calcified hematomas over pressure points such as the elbow) and spheroids (fat-containing cysts on forearms and shins) are also frequently seen. Hypotonia and delayed motor development may occur. The mutation that causes this type of EDS is in the genes COL5A1, COL5A2, and COL1A1. It involves the skin more than hEDS.

Vascular EDS (type 4 vEDS) is characterized by thin, translucent skin that is extremely fragile and bruises easily. Arteries and certain organs such as the intestines and uterus are also fragile and prone to rupture. People with this type typically have short stature, and thin scalp hair. It also has characteristic facial features including large eyes, an undersized chin, sunken cheeks, a thin nose and lips, and ears without lobes. Joint hypermobility is present, but generally confined to the small joints (fingers, toes). Other common features include club foot, tendon and/or muscle rupture, acrogeria (premature aging of the skin of the hands and feet), early onset varicose veins, pneumothorax (collapse of a lung), recession of the gums, and a decreased amount of fat under the skin. Is can be caused by the mutations in the COL3A1 gene.

Kyphoscoliosis EDS (type 6 kEDS) is associated with severe hypotonia at birth, delayed motor development, progressive scoliosis (present from birth), and scleral fragility. Affected people may also have easy bruising, fragile arteries that are prone to rupture, unusually small corneas, and osteopenia (low bone density). Other common features include a “marfanoid habitus” which is characterized by long, slender fingers (arachnodactyly), unusually long limbs, and a sunken chest (pectus excavatum) or protruding chest (pectus carinatum). It can be caused by mutations in the gene PLOD1.

Arthrochalasia EDS (types 7A & B aEDS) is characterized by severe joint hypermobility and congenital hip dislocation. Other common features include fragile, elastic skin with easy bruising, hypotonia, kyphoscoliosis (kyphosis and scoliosis), and mild osteopenia. Type-I collagen is usually affected. It is very rare, with about 30 cases reported. It is more severe than the hypermobility type. Mutations in the genes COL1A1 and COL1A2 cause it.

Dermatosparaxis EDS (type 7C dEDS) is associated with extremely fragile skin leading to severe bruising and scarring; saggy, redundant skin, especially on the face; and hernias. It is extremely rare, with around 10 cases reported.

Brittle cornea syndrome is characterized by thin corneaa, early-onset progressive keratoglobus or keratoconus, and blue sclerae. Classic symptoms, such as hypermobile joints and hyperelastic skin, are also seen often.

Classical-like EDS (type 1 cEDS) is characterized by skin hyperextensibility with velvety skin texture and absence of atrophic scarring, generalized joint hypermobility with or without recurrent dislocations (most often shoulder and ankle), and easily bruised skin or spontaneous ecchymoses (discolorations of the skin resulting from bleeding underneath).

Spondylodysplastic EDS (spEDS) is characterized by short stature (progressive in childhood), muscle hypotonia (ranging from severe congenital, to mild later-onset), and bowing of limbs.

Musculocontractural EDS (mcEDS) is characterized by congenital multiple contractures, characteristically adduction-flexion contractures and/or talipes equinovarus (clubfoot), characteristic craniofacial features, which are evident at birth or in early infancy, and skin features such as skin hyperextensibility, bruising, skin fragility with atrophic scars, and increased palmar wrinkling.

Myopathic EDS (mEDS) is characterized by congenital muscle hypotonia and/or muscle atrophy that improves with age, proximal joint contractures (joints of the knee, hip and elbow), and hypermobility of distal joints (joints of the ankles, wrists, feet and hands).

Periodontal EDS (pEDS) is characterized by severe and intractable periodontitis of early onset (childhood or adolescence), lack of attached gingiva, pretibial plaques, and family history of a first-degree relative who meets clinical criteria.

Cardiac-valvular EDS (cvEDS) is characterized by severe progressive cardiac-valvular problems (aortic valve, mitral valve), skin problems (hyperextensibility, atrophic scars, thin skin, easy bruising), and joint hypermobility (generalized or restricted to small joints).

### Vasculopathy

Vasculopathy is a term used to describe a disease affecting blood vessels. It often includes vascular abnormalities caused by degenerative, metabolic and inflammatory conditions, embolic diseases, coagulative disorders, and functional disorders such as posteri or reversible encephalopathy syndrome. The etiology of vasculopathy is generally unknown and the condition is frequently not pathologically proven. Vasculitis, on the other hand, is a more specific term and is defined as inflammation of the wall of a blood vessel.

Vascular Ehlers-Danlos Syndrome (vEDS)

Vascular Ehlers-Danlos Syndrome (vEDS) is an inherited connective tissue disorder caused by heterozygous mutations in the collagen type III alpha 1 chain (COL3A1) gene. The major cause of mortality in vEDS is arterial dissection and/or rupture, but the pathogenesis of this disease has not been established in the art. Effective treatment strategies for this devastating condition do not exist. The current belief is that reduced amounts of collagen III lead directly to the signs and symptoms of vEDS due to an inherent loss of structural integrity of the tissues. However, early pathogenic models of Marfan Syndrome (MFS) also singularly invoked tissue weakness imposed by failed elastogenesis, but subsequent work clearly demonstrated enhanced transforming growth factor beta (TGF-β) signaling in a mouse model deficient in fibrillin-1, the deficient gene product in MFS. Follow-up work went on to show that TGF-β and downstream cellular signaling molecules were major mediators of disease pathology. Further, therapies that attenuate TGF-β signaling and related pathways, such as TGF-β neutralizing antibody (Nab), the angiotensin-II (Ang-II) type 1 receptor blocker (ARB) losartan, or the inhibitor of ERK1/2 activation RDEA119/trametinib, can suppress aortic disease in MFS mice.

However, similar to other heritable vasculopathies such as Marfan syndrome and Loeys-Dietz syndrome, signaling abnormalities that are provided herein are major mediators of disease pathology in vEDS. RNA-seq profiling on the aortas of mice with patient-derived Col3a1 mutations demonstrated elevated PLC/IP3/PKC/ERK signaling compared to wild-type aortas. Immunoblotting of the proximal descending thoracic aorta confirmed elevated PKC and ERK1/2 activation.

In certain embodiments, COL3A1 comprises the following amino acid sequence (NCBI Accession No: AAH28178.1 (SEQ ID NO: 1), incorporated herein by reference in its entirety):

| | |
|---|---|
| 1 | MMSFVQKGSW LLLALLHPTI ILAQQEAVEG GCSHLGQSYA DRDVWKPEPC QICVCDSGSV |
| 61 | LCDDIICDDQ ELDCPNPEIP FGECCAVCPQ PPTAPTRPPN GQGPQGPKGD PGPPGIPGRN |
| 121 | GDPGIPGQPG SPGSPGPPGI CESCPTGPQN YSPQYDSYDV KSGVAVGGLA GYPGPAGPPG |
| 181 | PPGPPGTSGH PGSPGSPGYQ GPPGEPGQAG PSGPPGPPGA IGPSGPAGKD GESGRPGRPG |
| 241 | ERGLPGPPGI KGPAGIPGFP GMKGHRGFDG RNGEKGETGA PGLKGENGLP GENGAPGPMG |
| 301 | PRGAPGERGR PGLPGAAGAR GNDGARGSDG QPGPPGPPGT AGFPGSPGAK GEVGPAGSPG |
| 361 | SNGAPGQRGE PGPQGHAGAQ GPPGPPGING SPGGKGEMGP AGIPGAPGLM GARGPPGPAG |
| 421 | ANGAPGLRGG AGEPGKNGAK GEPGPRGERG EAGIPGVPGA KGEDGKDGSP GEPGANGLPG |
| 481 | AAGERGAPGF RGPAGPNGIP GEKGPAGERG APGPAGPRGA AGEPGRDGVP GGPGMRGMPG |
| 541 | SPGGPGSDGK PGPPGSQGES GRPGPPGPSG PRGQPGVMGF PGPKGNDGAP GKNGERGGPG |
| 601 | GPGPQGPPGK NGETGPQGPP GPTGPGGDKG DTGPPGPQGL QGLPGTGGPP GENGKPGEPG |
| 661 | PKGDAGAPGA PGGKGDAGAP GERGPPGLAG APGLRGGAGP PGPEGGKGAA GPPGPPGAAG |
| 721 | TPGLQGMPGE RGGLGSPGPK GDKGEPGGPG ADGVPGKDGP RGPTGPIGPP GPAGQPGDKG |
| 781 | EGGAPGLPGI AGPRGSPGER GETGPPGPAG FPGAPGQNGE PGGKGERGAP GEKGEGGPPG |
| 841 | VAGPPGKDGT SGHPGPIGPP GPRGNRGERG SEGSPGHPGQ PGPPGPPGAP GPCCGGVGAA |
| 901 | AIAGIGGEKA GGFAPYYGDE PMDFKINTDE IMTSLKSVNG QIESLISPDG SRKNPARNCR |
| 961 | DLKFCHPELK SGEYWVDPNQ GCKLDAIKVF CNMETGETCI SANPLNVPRK HWWTDSSAEK |
| 1021 | KHVWFGESMD GGFQFSYGNP ELPEDVLDVQ LAFLRLLSSR ASQNITYHCK NSIAYMDQAS |
| 1081 | GNVKKALKLM GSNEGEFKAE GNSKFTYTVL EDGCTKHTGE WSKTVFEYRT RKAVRLPIVD |
| 1141 | IAPYDIGGPD QEFGVDVGPV CFL |

In certain embodiments, COL3A1 comprises the following nucleic acid sequence, the start and stop codons are bold and underlined (NCBI Accession No: NM_000090.3 (SEQ ID NO: 2), incorporated herein by reference in its entirety):

| | |
|---|---|
| 1 | ggctgagttt tatgacgggc ccggtgctga agggcaggga acaacttgat ggtgctactt |
| 61 | tgaactgctt ttcttttctc ctttttgcac aaagagtctc atgtctgata tttagacatg |
| 121 | atgagctttg tgcaaaaggg gagctggcta cttctcgctc tgcttcatcc cactattatt |
| 181 | ttggcacaac aggaagctgt tgaaggagga tgttcccatc ttggtcagtc ctatgcggat |
| 241 | agagatgtct ggaagccaga accatgccaa atatgtgtct gtgactcagg atccgttctc |
| 301 | tgcgatgaca taatatgtga cgatcaagaa ttagactgcc ccaacccaga aattccattt |
| 361 | ggagaatgtt gtgcagtttg cccacagcct ccaactgctc ctactcgccc tcctaatggt |
| 421 | caaggacctc aaggccccaa gggagatcca ggccctcctg gtattcctgg gagaaatggt |
| 481 | gaccctggta ttccaggaca accagggtcc cctggttctc ctggcccccc tggaatctgt |
| 541 | gaatcatgcc ctactggtcc tcagaactat tctccccagt atgattcata tgatgtcaag |
| 601 | tctggagtag cagtaggagg actcgcaggc tatcctggac cagctggccc cccaggccct |
| 661 | cccggtcccc ctggtacatc tggtcatcct ggttcccctg gatctccagg ataccaagga |
| 721 | ccccctggtg aacctgggca agctggtcct tcaggccctc caggacctcc tggtgctata |
| 781 | ggtccatctg gtcctgctgg aaaagatgga gaatcaggta gacccggacg acctggagag |
| 841 | cgaggattgc ctggacctcc aggtatcaaa ggtccagctg ggatacctgg attccctggt |
| 901 | atgaaaggac acagaggctt cgatggacga aatggagaaa agggtgaaac aggtgctcct |
| 961 | ggattaaagg gtgaaaatgg tcttccaggc gaaaatggag ctcctggacc catgggtcca |
| 1021 | agaggggctc ctggtgagcg aggacggcca ggacttcctg gggctgcagg tgctcggggt |
| 1081 | aatgacggtg ctcgaggcag tgatggtcaa ccaggccctc ctggtcctcc tggaactgcc |
| 1141 | ggattccctg gatcccctgg tgctaagggt gaagttggac ctgcagggtc tcctggttca |
| 1201 | aatggtgccc ctggacaaag aggagaacct ggacctcagg gacacgctgg tgctcaaggt |
| 1261 | cctcctggcc ctcctgggat taatggtagt cctggtggta aaggcgaaat gggtcccgct |
| 1321 | ggcattcctg gagctcctgg actgatggga gcccggggtc ctccaggacc agccggtgct |
| 1381 | aatggtgctc ctggactgcg aggtggtgca ggtgagcctg gtaagaatgg tgccaaagga |
| 1441 | gagcccggac cacgtggtga acgcggtgag gctggtattc caggtgttcc aggagctaaa |
| 1501 | ggcgaagatg gcaaggatgg atcacctgga gaacctggtg caaatgggct tccaggagct |
| 1561 | gcaggagaaa ggggtgcccc tgggttccga ggacctgctg gaccaaatgg catcccagga |
| 1621 | gaaaagggtc ctgctggaga gcgtggtgct ccaggccctg cagggcccag aggagctgct |
| 1681 | ggagaacctg gcagagatgg cgtccctgga ggtccaggaa tgaggggcat gcccggaagt |
| 1741 | ccaggaggac caggaagtga tgggaaacca gggcctcccg gaagtcaagg agaaagtggt |
| 1801 | cgaccaggtc ctcctgggcc atctggtccc cgaggtcagc ctggtgtcat gggcttcccc |
| 1861 | ggtcctaaag gaaatgatgg tgctcctggt aagaatggag aacgaggtgg ccctggagga |
| 1921 | cctggccctc agggtcctcc tggaaagaat ggtgaaactg gacctcaggg acccccaggg |
| 1981 | cctactgggc ctggtggtga caaaggagac acaggacccc ctggtccaca aggattacaa |
| 2041 | ggcttgcctg gtacaggtgg tcctccagga gaaaatggaa aacctgggga accaggtcca |
| 2101 | aagggtgatg ccggtgcacc tggagctcca ggaggcaagg gtgatgctgg tgcccctggt |
| 2161 | gaacgtggac ctcctggatt ggcaggggcc ccaggactta gaggtggagc tggtccccct |
| 2221 | ggtcccgaag gaggaaaggg tgctgctggt cctcctgggc cacctggtgc tgctggtact |
| 2281 | cctggtctgc aaggaatgcc tggagaaaga ggaggtcttg gaagtcctgg tccaaagggt |
| 2341 | gacaagggtg aaccaggcgg tccaggtgct gatggtgtcc cagggaaaga tggcccaagg |
| 2401 | ggtcctactg gtcctattgg tcctcctggc ccagctggcc agcctggaga taagggtgaa |
| 2461 | ggtggtgccc ccggacttcc aggtatagct ggacctcgtg gtagccctgg tgagagaggt |
| 2521 | gaaactggcc ctccaggacc tgctggtttc cctggtgctc ctggacagaa tggtgaacct |
| 2581 | ggtggtaaag gagaaagagg ggctccgggt gagaaaggtg aaggaggccc tcctggagtt |
| 2641 | gcaggacccc ctggaggttc tggacctgct ggtcctcctg gtccccaagg tgtcaaaggt |
| 2701 | gaacgtggca gtcctggtgg acctggtgct gctggcttcc ctggtgctcg tggtcttcct |
| 2761 | ggtcctcctg gtagtaatgg taacccagga cccccaggtc ccagcggttc tccaggcaag |
| 2821 | gatgggcccc caggtcctgc gggtaacact ggtgctcctg gcagccctgg agtgtctgga |
| 2881 | ccaaaaggtg atgctggcca accaggagag aagggatcgc ctggtgccca gggcccacca |
| 2941 | ggagctccag gcccacttgg gattgctggg atcactggag cacggggtct tgcaggacca |
| 3001 | ccaggcatgc caggtcctag gggaagccct ggccctcagg gtgtcaaggg tgaaagtggg |
| 3061 | aaaccaggag ctaacggtct cagtggagaa cgtggtcccc ctggacccca gggtcttcct |
| 3121 | ggtctggctg gtacagctgg tgaacctgga agagatggaa accctggatc agatggtctt |
| 3181 | ccaggccgag atggatctcc tggtggcaag ggtgatcgtg gtgaaaatgg ctctcctggt |
| 3241 | gcccctggcg ctcctggtca tccaggccca cctggtcctg tcggtccagc tggaaagagt |
| 3301 | ggtgacagag gagaaagtgg ccctgctggc cctgctggtg ctcccggtcc tgctggttcc |
| 3361 | cgaggtgctc ctggtcctca aggcccacgt ggtgacaaag gtgaaacagg tgaacgtgga |
| 3421 | gctgctggca tcaaaggaca tcgaggattc cctggtaatc caggtgcccc aggttctcca |
| 3481 | ggccctgctg gtcagcaggg tgcaatcggc agtccaggac ctgcaggccc cagaggacct |
| 3541 | gttggaccca gtggacctcc tggcaaagat ggaaccagtg gacatccagg tcccattgga |
| 3601 | ccaccagggc ctcgaggtaa cagaggtgaa agaggatctg agggctcccc aggccaccca |
| 3661 | gggcaaccag gccctcctgg acctcctggt gcccctggtc cttgctgtgg tggtgttgga |
| 3721 | gccgctgcca ttgctgggat tggaggtgaa aaagctggcg gttttgcccc gtattatgga |
| 3781 | gatgaaccaa tggatttcaa aatcaacacc gatgagatta tgacttcact caagtctgtt |
| 3841 | aatggacaaa tagaaagcct cattagtcct gatggttctc gtaaaaaccc cgctagaaac |
| 3901 | tgcagagacc tgaaattctg ccatcctgaa ctcaagagtg gagaatactg ggttgaccct |
| 3961 | aaccaaggat gcaaattgga tgctatcaag gtattctgta atatggaaac tggggaaaca |
| 4021 | tgcataagtg ccaatccttt gaatgttcca cggaaacact ggtggacaga ttctagtgct |
| 4081 | gagaagaaac acgtttggtt tggagagtcc atggatggtg gttttcagtt tagctacggc |
| 4141 | aatcctgaac ttcctgaaga tgtccttgat gtgcagctgg cattccttcg acttctctcc |
| 4201 | agccgagctt cccagaacat cacatatcac tgcaaaaata gcattgcata catggatcag |
| 4261 | gccagtggaa atgtaaagaa ggccctgaag ctgatggggt caaatgaagg tgaattcaag |
| 4321 | gctgaaggaa atagcaaatt cacctacaca gttctggagg atggttgcac gaaacacact |
| 4381 | ggggaatgga gcaaaacagt ctttgaatat cgaacacgca aggctgtgag actacctatt |
| 4441 | gtagatattg caccctatga cattggtggt cctgatcaag aatttggtgt ggacgttggc |
| 4501 | cctgtttgct ttttataaac caaactctat ctgaaatccc aacaaaaaaa atttaactcc |
| 4561 | atatgtgttc ctcttgttct aatcttgtca accagtgcaa gtgaccgaca aaattccagt |
| 4621 | tatttatttc caaaatgttt ggaaacagta taatttgaca aagaaaaatg atacttctct |
| 4681 | ttttttgctg ttccaccaaa tacaattcaa atgcttttt ttttattttt ttaccaattc |
| 4741 | caatttcaaa atgtctcaat ggtgctataa taaataaact tcaacactct ttatgataac |
| 4801 | aacactgtgt tatattcttt gaatcctagc ccatctgcag agcaatgact gtgctcacca |
| 4861 | gtaaaagata acctttcttt ctgaaatagt caaatacgaa attagaaaag cccccctat |
| 4921 | tttaactacc tcaactggtc agaaacacag attgtattct atgagtccca gaagatgaaa |
| 4981 | aaaattttat acgttgataa aacttataaa tttcattgat taatctcctg gaagattggt |
| 5041 | ttaaaaagaa aagtgtaatg caagaattta aagaaatatt tttaaagcca caattatttt |
| 5101 | aatattggat atcaactgct tgtaaaggtg ctcctctttt ttcttgtcat tgctggtcaa |
| 5161 | gattactaat atttgggaag gctttaaaga cgcatgttat ggtgctaatg tactttcact |
| 5221 | tttaaactct agatcagaat tgttgacttg cattcagaac ataaatgcac aaaatctgta |
| 5281 | catgtctccc atcagaaaga ttcattggca tgccacaggg gattctcctc cttcatcctg |
| 5341 | taaaggtcaa caataaaaac caaattatgg ggctgctttt gtcacactag catagagaat |
| 5401 | gtgttgaaat ttaactttgt aagcttgtat gtggttgttg atcttttttt tccttacaga |
| 5461 | cacccataat aaaatatcat attaaaattc |

### MEK Inhibitors

In embodiments, the disclosure provides methods for treating vasculopathies (e.g., vEDS) in a subject in need thereof, the methods comprising administering to the subject a pharmaceutical composition comprising a therapeutically effective amount of an agent that decreases the activity or expression of ERK or PKC. For example, a Ras/Raf/MEK/ERK pathway inhibitor. In embodiments, the Ras pathway inhibitor is selected from a Raf inhibitor such as vemurafenib, sorafenib, or dabrafenib, a MEK inhibitor such as AZD6244 (Selumetinib), PD0325901, GSK1120212 (Trametinib), U0126-EtOH, PD184352, RDEA119 (Rafametinib), PD98059, BIX 02189, MEK162 (Binimetinib), AS-703026 (Pimasertib), SL-327, BIX02188, AZD8330, TAK-733, cobimetinib or PD318088, and an ERK inhibitor such as LY3214996, BVD-523 or GDC-0994.

In embodiments, the MEK inhibitor is selected from the group consisting of Trametinib, Refametinib, Cobimetinib, TAK-733, PD0325901, PD184352 (CI-10-40), R05126766, RO-4987655; E6201; GDC-0623; CH5126766; G-573; WX-554; Selumetinib, Binimetinib and Pimasertib. In embodiments, the MEK inhibitor comprises cobimetinib. In embodiments, the MEK inhibitor comprises trametinib. In embodiments, the MEK inhibitor comprises trametinib and cobimetinib. In embodiments the MEK inhibitors, or pharmaceutically acceptable salts thereof are also contemplated herewith.

In certain embodiments, the MEK inhibitor can be administered in a concentration from about 0.001 mg/kg to about 250 mg/kg body weight, e.g., 0.001 mg/kg, 0.05 mg/kg 0.01 mg/kg, 0.05 mg/kg, 1 mg/kg, 5 mg/kg, 10 mg/kg, 25 mg/kg, 50 mg/kg, 75 mg/kg, 100 mg/kg, 125 mg/kg, 150 mg/kg, 175 mg/kg, 200 mg/kg, 225 mg/kg, or 250 mg/kg body weight.

### Protein Kinase C (PKC) Inhibitors

Protein kinase C, commonly abbreviated to PKC, is a family of protein kinase enzymes that are involved in controlling the function of other proteins through the phosphorylation of hydroxyl groups of serine and threonine amino acid residues on these proteins, or a member of this family. PKC enzymes in turn are activated by signals such as increases in the concentration of diacylglycerol (DAG) or calcium ions (Ca²⁺). Thus, PKC enzymes play important roles in several signal transduction cascades. The PKC family consists of fifteen isozymes in humans, and are divided into three subfamilies, based on their second messenger requirements: conventional (or classical), novel, and atypical. Conventional PKCs contain the isoforms α, β_{I}, β_{II}, and γ. These require Ca²⁺, DAG, and a phospholipid such as phosphatidylserine for activation. Novel (n) PKCs include the δ, ε, η, and θ isoforms, and require DAG, but do not require Ca²⁺ for activation. Thus, conventional and novel PKCs are activated through the same signal transduction pathway as phospholipase C. On the other hand, atypical (a) PKCs (including protein kinase Mζ and τ/λ isoforms) require neither Ca²⁺ nor diacylglycerol for activation. The term “protein kinase C” as used herein generally refers to the entire family of isoforms.

Exemplary PKC agents include, but are not limited to ruboxistaurin, chelerythrine, miyabenol C, myricitrin, gossypol, verbascoside, BIM-1, or Bryostatin 1. In embodiments, the PKC inhibitor comprises enzastaurin. In embodiments, the PKC inhibitor comprises ruboxistaurin. In embodiments, the PKC agents, or pharmaceutically acceptable salts thereof are also contemplated herewith.

In certain embodiments, the agent that decreases the activity or expression of PKC can be administered in a concentration from about 0.001 mg/kg to about 250 mg/kg body weight, e.g., 0.001 mg/kg, 0.05 mg/kg 0.01 mg/kg, 0.05 mg/kg, 1 mg/kg, 5 mg/kg, 10 mg/kg, 25 mg/kg, 50 mg/kg, 75 mg/kg, 100 mg/kg, 125 mg/kg, 150 mg/kg, 175 mg/kg, 200 mg/kg, 225 mg/kg, or 250 mg/kg body weight.

In certain embodiments, PKC comprises the following amino acid sequence (NCBI Accession No: NP 002728.1, incorporated herein by reference in its entirety):

| | |
|---|---|
| 1 | MADVFPGNDS TASQDVANRF ARKGALRQKN VHEVKDHKFI ARFFKQPTFC SHCTDFIWGF |
| 61 | GKQGFQCQVC CFVVHKRCHE FVTFSCPGAD KGPDTDDPRS KHKFKIHTYG SPTFCDHCGS |
| 121 | LLYGLIHQGM KCDTCDMNVH KQCVINVPSL CGMDHTEKRG RIYLKAEVAD EKLHVTVRDA |
| 181 | KNLIPMDPNG LSDPYVKLKL IPDPKNESKQ KTKTIRSTLN PQWNESFTFK LKPSDKDRRL |
| 241 | SVEIWDWDRT TRNDFMGSLS FGVSELMKMP ASGWYKLLNQ EEGEYYNVPI PEGDEEGNME |
| 301 | LRQKFEKAKL GPAGNKVISP SEDRKQPSNN LDRVKLTDFN FLMVLGKGSF GKVMLADRKG |
| 361 | TEELYAIKIL KKDVVIQDDD VECTMVEKRV LALLDKPPFL TQLHSCFQTV DRLYFVMEYV |
| 421 | NGGDLMYHIQ QVGKFKEPQA VFYAAEISIG LFFLHKRGII YRDLKLDNVM LDSEGHIKIA |
| 481 | DFGMCKEHMM DGVTTRTFCG TPDYIAPEII AYQPYGKSVD WWAYGVLLYE MLAGQPPFDG |
| 541 | EDEDELFQSI MEHNVSYPKS LSKEAVSICK GLMTKHPAKR LGCGPEGERD VREHAFFRRI |
| 601 | DWEKLENREI QPPFKPKVCG KGAENFDKFF TRGQPVLTPP DQLVIANIDQ SDFEGFSYVN |
| 661 | PQFVHPILQS AV |

In certain embodiments, the PKC comprises the following nucleotide sequence, the coding sequence is bold and underlined (NCBI Accession No: NM_002737.2, incorporated herein by reference in its entirety):

| | |
|---|---|
| 1 | ggccgcagct ccccggcgga ggcaagaggt ggttgggggg gaccatggct gacgttttcc |
| 61 | cgggcaacga ctccacggcg tctcaggacg tggccaaccg cttcgcccgc aaaggggcgc |
| 121 | tgaggcagaa gaacgtgcac gaggtgaagg accacaaatt catcgcgcgc ttcttcaagc |
| 181 | agcccacctt ctgcagccac tgcaccgact tcatctgggg gtttgggaaa caaggcttcc |
| 241 | agtgccaagt ttgctgtttt gtggtccaca agaggtgcca tgaatttgtt actttttctt |
| 301 | gtccgggtgc ggataaggga cccgacactg atgaccccag gagcaagcac aagttcaaaa |
| 361 | tccacactta cggaagcccc accttctgcg atcactgtgg gtcactgctc tatggactta |
| 421 | tccatcaagg gatgaaatgt gacacctgcg atatgaacgt tcacaagcaa tgcgtcatca |
| 481 | atgtccccag cctctgcgga atggatcaca ctgagaagag ggggcggatt tacctaaagg |
| 541 | ctgaggttgc tgatgaaaag ctccatgtca cagtacgaga tgcaaaaaat ctaatcccta |
| 601 | tggatccaaa cgggctttca gatccttatg tgaagctgaa acttattcct gatcccaaga |
| 661 | atgaaagcaa gcaaaaaacc aaaaccatcc gctccacact aaatccgcag tggaatgagt |
| 721 | cctttacatt caaattgaaa ccttcagaca aagaccgacg actgtctgta gaaatctggg |
| 781 | actgggatcg aacaacaagg aatgacttca tgggatccct ttcctttgga gtttcggagc |
| 841 | tgatgaagat gccggccagt ggatggtaca agttgcttaa ccaagaagaa ggtgagtact |
| 901 | acaacgtacc cattccggaa ggggacgagg aaggaaacat ggaactcagg cagaaattcg |
| 961 | agaaagccaa acttggccct gctggcaaca aagtcatcag tccctctgaa gacaggaaac |
| 1021 | aaccttccaa caaccttgac cgagtgaaac tcacggactt caatttcctc atggtgttgg |
| 1081 | gaaaggggag ttttggaaag gtgatgcttg ccgacaggaa gggcacagaa gaactgtatg |
| 1141 | caatcaaaat cctgaagaag gatgtggtga ttcaggatga tgacgtggag tgcaccatgg |
| 1201 | tagaaaagcg agtcttggcc ctgcttgaca aacccccgtt cttgacgcag ctgcactcct |
| 1261 | gcttccagac agtggatcgg ctgtacttcg tcatggaata tgtcaacggt ggggacctca |
| 1321 | tgtaccacat tcagcaagta ggaaaattta aggaaccaca agcagtattc tatgcggcag |
| 1381 | agatttccat cggattgttc tttcttcata aaagaggaat catttatagg gatctgaagt |
| 1441 | tagataacgt catgttggat tcagaaggac atatcaaaat tgctgacttt gggatgtgca |
| 1501 | aggaacacat gatggatgga gtcacgacca ggaccttctg tgggactcca gattatatcg |
| 1561 | ccccagagat aatcgcttat cagccgtatg gaaaatctgt ggactggtgg gcctatggcg |
| 1621 | tcctgttgta tgaaatgctt gccgggcagc ctccatttga tggtgaagat gaagacgagc |
| 1681 | tatttcagtc tatcatggag cacaacgttt cctatccaaa atccttgtcc aaggaggctg |
| 1741 | tttctatctg caaaggactg atgaccaaac acccagccaa gcggctgggc tgtgggcctg |
| 1801 | agggggagag ggacgtgaga gagcatgcct tcttccggag gatcgactgg gaaaaactgg |
| 1861 | agaacaggga gatccagcca ccattcaagc ccaaagtgtg tggcaaagga gcagagaact |
| 1921 | ttgacaagtt cttcacacga ggacagcccg tcttaacacc acctgatcag ctggttattg |
| 1981 | ctaacataga ccagtctgat tttgaagggt tctcgtatgt caacccccag tttgtgcacc |
| 2041 | ccatcttaca gagtgcagta tgaaactcac cagcgagaac aaacacctcc ccagccccca |
| 2101 | gccctccccg cagtgggaag tgaatcctta accctaaaat tttaaggcca cggccttgtg |
| 2161 | tctgattcca tatggaggcc tgaaaattgt agggttatta gtccaaatgt gatcaactgt |
| 2221 | tcagggtctc tctcttacaa ccaagaacat tatcttagtg gaagatggta cgtcatgctc |
| 2281 | agtgtccagt ttaattctgt agaagttacg tctggctcta ggttaaccct tcctagaaag |
| 2341 | caagcagact gttgccccat tttgggtaca atttgatata ctttccatac cctccatctg |
| 2401 | tggatttttc agcattggaa tcccccaacc agagatgtta aagtgagcct gtcccaggaa |
| 2461 | acatctccac ccaagacgtc tttggaatcc aagaacagga agccaagaga gtgagcaggg |
| 2521 | agggattggg ggtgggggag gcctcaaaat accgactgcg tccattctct gcctccatgg |
| 2581 | aaacagcccc tagaatctga aaggccggga taaacctaat cactgttccc aaacattgac |
| 2641 | aaatcctaac ccaaccatgg tccagcagtt accagtttaa acaaaaaaac ctcagatgag |
| 2701 | tgttgggtga atctgtcatc tggtaccctc cttggttgat aactgtcttg atacttttca |
| 2761 | ttctttgtaa gaggccaaat cgtctaagga cgttgctgaa caagcgtgtg aaatcatttc |
| 2821 | agatcaagga taagccagtg tgtacatatg ttcattttaa tctctgggag attatttttc |
| 2881 | catccagggt gccatcagta atcatgccac tactcaccag tgttgttcgc caacacccac |
| 2941 | ccccacacac accaacattt tgctgcctac cttgttatcc ttctcaagaa gctgaagtgt |
| 3001 | acgccctctc cccttttgtg cttatttatt taataggctg cagtgtcgct tatgaaagta |
| 3061 | cgatgtacag taacttaatg gaagtgctga ctctagcatc agcctctacc gattgatttt |
| 3121 | cctcccttct ctagccctgg atgtccactt agggataaaa agaatatggt tttggttccc |
| 3181 | atttctagtt cacgttgaat gacaggcctg gagctgtaga atcaggaaac ccggatgcct |
| 3241 | aacagctcaa agatgttttg ttaatagaag gattttaata cgttttgcaa atgcatcatg |
| 3301 | caatgaattt tgcatgttta taataaacct taataacaag tgaatctata ttattgatat |
| 3361 | aatcgtatca agtataaaga gagtattata ataattttat aagacacaat tgtgctctat |
| 3421 | ttgtgcaggt tcttgtttct aatcctcttt tctaattaag ttttagctga atcccttgct |
| 3481 | tctgtgcttt ccctccctgc acatgggcac tgtatcagat agattacttt ttaaatgtag |
| 3541 | ataaaatttc aaaaatgaat ggctagttta cgtgatagat taggctctta ctacatatgt |
| 3601 | gtgtgtatat atatgtattt gattctacct gcaaacaaat ttttattggt gaggactatt |
| 3661 | tttgagctga cactccctct tagtttcttc atgtcacctt tcgtcctggt tcctccgcca |
| 3721 | ctcttcctct tggggacaac aggaagtgtc tgattccagt ctgcctagta cgttggtaca |
| 3781 | cacgtggcat tgccgcagca cctgggctga cctttgtgtg tgcgtgtgtg tgtgtttcct |
| 3841 | tcttcccttc agcctgtgac tgttgctgac tccaggggtg ggagggatgg ggagactccc |
| 3901 | ctcttgctgt gtgtactgga cacgcaggaa gcatgctgtc ttgctgcctc tgcaacgacc |
| 3961 | tgtcgtttgc tccagcatgc acaaacttcg tgagaccaac acagccgtgc cctgcaggca |
| 4021 | ccagcacgtg cttttcagag gctgcggact ttcttccagc cattgtggca ttggcctttc |
| 4081 | cagtcttggg aggagcgcgc tgctttggtg agacaccccc atgcaaggtc ctcagagtag |
| 4141 | ccgggttcta ccacaaacag aaacagaatg aaagtagctg tcagtccttg tagagagccg |
| 4201 | ctctgtttcc tcccagaagc atctcccagc taagctcgca ttatttttct cctctggctg |
| 4261 | tttgcctgaa gttcacagaa cacacaacca tgaaaggctt tttgaggtga gaggcccagg |
| 4321 | tggtcctggc aaccctgagt agaaggagag acggggtagg gaacgggccc ggccagaaaa |
| 4381 | gaaccatttc ttctgccatc ttttatgcac catagacatc gagactccag ggggtcctgg |
| 4441 | ctcccctgtc cctgcagccc tgcaggtcag tgcatgatct gggttcgtgt cctgaccagg |
| 4501 | tgctcctcct ttgatccgag gggaaaggga ctggtttata gaaagagcct aggagacaaa |
| 4561 | agggccagtc cccctgccca gaatggagca gcagcaggac agacccccac gaggcccccc |
| 4621 | agagaggagg aagatcccac ggaggaacac atgaggttag ggacccttgt tcagcacccc |
| 4681 | aaacagcctg cctgtttaaa gcaggcagca ggcttaggcc ttccctgcaa ccccaacacc |
| 4741 | cacaagtttg tttctctagg aaacacattc actgtctcag ctggctgtta ctctctcaga |
| 4801 | ccatatggca aagttttcca agaaaatgcc ccgacagggg tgcccagcac actgcctgag |
| 4861 | ggacaacaga catcagaaca aacccccaga gagaaacagt caaaatcagg gcccggtgca |
| 4921 | gtgttgtcat gtggaacctg ctttatccat tgctgagtgt tgaatgtggg taatggttag |
| 4981 | ggctttccag atctcagcag ccaaagacag ttattgttgg aagactgtca tgtagataac |
| 5041 | catgagcaat ggctcgcctc agaatcagtt cataaaattc tatggtactg gccccttcgt |
| 5101 | gggtattgtg tgaaatgaga tggtggcgag gggtgcgctg tggaactgcc gcagccacgc |
| 5161 | aggaggtccc tgggggatgc tttgggaagt ccttgcccct gagcactgcc tgattgccag |
| 5221 | ggcctgtgga ggtctaggcc gcctggcaga atctagcacc gtccgaatcc ccgcaggacc |
| 5281 | catggagcta tgaccacacc aggccattca aatggctctg cattatcttc ccttggaagg |
| 5341 | tggccactcc tcggtggcag ggcctttccc tgaggctgca ggccgtgggc tggcagcccg |
| 5401 | tctcttggca tttcaattga aggtcaccag gtgctgggtt tgaaaggaag tcactggagt |
| 5461 | gctgccaggg gccgccctcc aaggttaatg agaggcccac atccaggcaa gaactaattc |
| 5521 | aaaaggcaga tcagaaacca caggagtcaa aattattgct ccggcagtgc ttcccttcct |
| 5581 | ttcatccact ggcctcgtgt ggtccatgca gggccactgt ctgccctttc tgatgccacg |
| 5641 | tattaggctt tcttactcag aattttgata gaaaaccatg gggccaagag ctctggaagc |
| 5701 | ctggccggaa agaccaaggt tcatgcagcc caacaaatga ttgttgagca cctctcggag |
| 5761 | ccaaagtcct taggcgagtg tggtgacttc ctggaaggag gatgcagact tccagagagc |
| 5821 | ccccccaacg gacgtgctga gaagggagag ggaggcgggg gctgtagtca ggaaggagcc |
| 5881 | agagaagaac agggtttggg tgcatccaga aatatgcctg cagtaggagg gagaggaagg |
| 5941 | ggtgccaccg tcaacggctt cccatcggag gtggttggtg cagatggaag tttctgtctg |
| 6001 | ctggccctca agagagtgtt ttgccaggga cacagtctgt tcctcctcag aaaacacccc |
| 6061 | ccaaatgcta acaacatccc caccagctgc tagaagcccc tttcccctcc ccaccttgaa |
| 6121 | gtagctcata gttctctggg cagagccaga ccatccagtg taccccagag gccagtaggt |
| 6181 | tcctgcccat tttcctctct ggcttcctgc caagaattat ggcagctgag gatgaatgga |
| 6241 | gaagtaaaaa caactaacac cgcacaacta acaactaaca ccgcagttcc cacctgggtt |
| 6301 | ccacttagca ggagacattt cggagggttt tttttgtttt tgttcctgtt tttttttttt |
| 6361 | ttgctggaat ttgttttctc agtactgaaa agagaaaaag tgacaatctt gtatttttaa |
| 6421 | aagcctcgga aaggtgatac catctgacag tcattttctc acgttggtct tctaaagtca |
| 6481 | cctatttctt gtgtgtgcac atcacaccat ttcctgtttc tttataaccc gacaagggta |
| 6541 | ggagtgcctg tttcccctgc tgggcacacc agacaatcgt aatcacaaaa cagacactga |
| 6601 | gccaggggcc caaagggtgt gatcatgaga gttaccggga cagcagtagg catgacagtc |
| 6661 | accaggaagg acaagggtgc tctgttgtta gtggccacac accaatttga caaggagtgt |
| 6721 | tgcgaaattt ttatttattt atttatttat tttgagatgg agtttcactc ttgttgccca |
| 6781 | ggctggagtg cggtggtaca atctcggctc actgcaacct ccacctccca ggttcaagcg |
| 6841 | attctcctgc ctcagcctcc caagtacctg ggactacagg tgcgtgccac cacacccagc |
| 6901 | taaattttgt gtttttagta gagatggggt ttcaccatgt tggccaggat ggtcttgaac |
| 6961 | ccctgacctc atgatctgcc tgcctcggcc tcccaaagtg ctgggattac aggcatgagc |
| 7021 | caccacgccc agccaaaata tttttttaaa gtcattttcc ttaagctgct tgggctacat |
| 7081 | gtgaaataca ctggacggtc aacattcctg tctcctccca tttgggctga tgcagcagat |
| 7141 | ccagggaatg ttacctgttt ctgctgctag aagatccagg aaattgggaa ggttacctga |
| 7201 | cgcacacatg gatgaaggcc atcatctaga aatggggtca accacaattg tgttaattcc |
| 7261 | gtagtgtcag ggattcttcg ggaaggtcaa cagtatgaag gattctgacc cctgtgcctc |
| 7321 | ccatttatgt gatcaggtga cagttaataa ccgtggaggt cacactcagc catccaacag |
| 7381 | ccttacagtg accctacaca aaagccccca aattccaaag actttttctt aacctaaagg |
| 7441 | aagaaattat ttgttaattc cagtagagca actgaatata ctgggctatt tgtacttttt |
| 7501 | tatagagaac tttaataata attctttaaa aatgagtttt tagaacaaag caactgacga |
| 7561 | tttcctaaga ttccaatgcc ctggagcttg taggaggact tagcctgggt cagctggagc |
| 7621 | acccccgacc tgatctccca ctgccagatt ttcccatgct cctagggtat ggagtccacg |
| 7681 | tgggaatgac tgcaagttca ggtggaactt ggccgactga tgctctgcga gtttttaata |
| 7741 | gacactgggg acaactgctt aaggtttaga aacttccaaa ccacaggaaa gacattttta |
| 7801 | gtgtccccca tccagaggca gccctggaat aggattccca ggggtttctg ggaccccttt |
| 7861 | ccttgctccg tgaggctctg tggccatctt ttggcaggag gaggatgctt ccttggctct |
| 7921 | gtgcccagac ccgcctggtc cccaggtctc tcaccttggg tgaagattca gagatgccct |
| 7981 | gtaaggattt tgcccactgg gcaactcaga aatacttcga tctcccaaga tataagaggc |
| 8041 | agcagcaaac gtgcctattg acgtctgttt catagttacc acttacgcga gtagacagaa |
| 8101 | ctcggctttt cagaaaatag gtgtcaagtc cactttataa gaaccttttt ttctaaaata |
| 8161 | agataaaagg tggctttgca ttttctgatt aaacgactgt gtctttgtca cctctgctta |
| 8221 | actttaggag tatccattcc tgtgattgta gacttttgtt gatattcttc ctggaagaat |
| 8281 | atcattcttt tttgaaggg ttggtttact agaatattca aaatcaatca tgaaggcagt |
| 8341 | tactattttg agtctaaagg ttttctaaaa attaacctca catcccttct gttagggtct |
| 8401 | ttcagaatat cttttataaa cagaagcatt tgaagtcatt gcttttgcta catgatttgt |
| 8461 | gtgtgtgaag gacataccac gtttaaatca ttaattgaaa aacatcatat aagccccaac |
| 8521 | tttgtttgga ggaagagacg gaggttgagg tttttccttc tgtataagca cctactgaca |
| 8581 | aaatgtagag gccattcaac cgtcaaacac catttggtta tatcgcagag gagacggatg |
| 8641 | tgtaaattac tgcattgctt tttttttcag tttgtataac ctctaatctc cgtttgcatg |
| 8701 | atacgctttg ttagaaacat taattgtagt ttggaagcaa gtgtgtatga ataaagataa |
| 8761 | tgatcattcc aaaaaaaaaa aaaaaaa |

### Gene Editing Agents

Compositions of the disclosure include at least one gene editing agent, comprising CRISPR-associated nucleases such as Cas9 and Cas12a gRNAs, Argonaute family of endonucleases, clustered regularly interspaced short palindromic repeat (CRISPR) nucleases, zinc-finger nucleases (ZFNs), transcription activator-like effector nucleases (TALENs), meganucleases, other endo- or exo-nucleases, or combinations thereof.

In recent years, several systems for targeting endogenous genes have been developed including homing endonucleases (HE) or meganucleases, zinc finger nucleases (ZFN), transcription activator-like effector nucleases (TALEN) and most recently clustered regularly interspaced short palindromic repeats (CRISPR)-associated system 9 (Cas9) proteins which utilize site-specific double-strand DNA break (DSB)-mediated DNA repair mechanisms. These enzymes induce a precise and efficient genome cutting through DSB-mediated DNS repair mechanisms. These DSB-mediated genome editing techniques enable target gene deletion, insertion, or modification.

In the past years, ZFNs and TALENs have revolutionized genome editing. The major drawbacks for ZFNs and TALENs are the uncontrollable off-target effects and the tedious and expensive engineering of custom DNA-binding fusion protein for each target site, which limit the universal application and clinical safety.

CRISPR (Clustered Regularly Interspaced Short Palindromic Repeats) is found in bacteria and is believed to protect the bacteria from phage infection. It has recently been used as a means to alter gene expression in eukaryotic DNA to introduce insertions or deletions as a way of increasing or decreasing transcription in the DNA of a targeted cell or population of cells. See for example, Horvath et al., Science (2010) 327:167-170; Terns et al., Current Opinion in Microbiology (2011) 14:321-327; Bhaya et al., Annu Rev Genet (2011) 45:273-297; Wiedenheft et al., Nature (2012) 482:331-338); Jinek M et al., Science (2012) 337:816-821; Cong L et al., Science (2013) 339:819-823; Jinek M et al., (2013) eLife 2:e00471; Mali P et al. (2013) Science 339:823-826; Qi L S et al. (2013) Cell 152:1173-1183; Gilbert L A et al. (2013) Cell 154:442-451; Yang H et al. (2013) Cell 154:1370-1379; and Wang H et al. (2013) Cell 153:910-918).

CRISPR methodologies employ a nuclease, CRISPR-associated (Cas), that complexes with small RNAs as guides (gRNAs) to cleave DNA in a sequence-specific manner upstream of the protospacer adjacent motif (PAM) in any genomic location. CRISPR may use separate guide RNAs known as the crRNA and tracrRNA. These two separate RNAs have been combined into a single RNA to enable site-specific mammalian genome cutting through the design of a short guide RNA. Cas and guide RNA (gRNA) may be synthesized by known methods. Cas/guide-RNA (gRNA) uses a non-specific DNA cleavage protein Cas, and an RNA oligonucleotide to hybridize to target and recruit the Cas/gRNA complex. See Chang et al., 2013, Cell Res. 23:465-472; Hwang et al., 2013, Nat. Biotechnol. 31:227-229; Xiao et al., 2013, Nucl. Acids Res. 1-11.

In general, the CRISPR/Cas proteins comprise at least one RNA recognition and/or RNA binding domain. RNA recognition and/or RNA binding domains interact with guide RNAs. CRISPR/Cas proteins can also comprise nuclease domains (i.e., DNase or RNase domains), DNA binding domains, helicase domains, RNase domains, protein-protein interaction domains, dimerization domains, as well as other domains.

CRISPR methodologies employ a nuclease, CRISPR-associated (Cas), that complexes with small RNAs as guides (gRNAs) to cleave DNA in a sequence-specific manner upstream of the protospacer adjacent motif (PAM) in any genomic location. CRISPR may use separate guide RNAs known as the crRNA and tracrRNA. These two separate RNAs have been combined into a single RNA to enable site-specific mammalian genome cutting through the design of a short guide RNA. Cas and guide RNA (gRNA) may be synthesized by known methods. Cas/guide-RNA (gRNA) uses a non-specific DNA cleavage protein Cas, and an RNA oligonucleotide to hybridize to target and recruit the Cas/gRNA complex. See Chang et al., 2013, Cell Res. 23:465-472; Hwang et al., 2013, Nat. Biotechnol. 31:227-229; Xiao et al., 2013, Nucl. Acids Res. 1-11.

The RNA-guided Cas9 biotechnology induces genome editing without detectable off-target effects. This technique takes advantage of the genome defense mechanisms in bacteria that CRISPR/Cas loci encode RNA-guided adaptive immune systems against mobile genetic elements (viruses, transposable elements and conjugative plasmids). Three types (I-III) of CRISPR systems have been identified. CRISPR clusters contain spacers, the sequences complementary to antecedent mobile elements. CRISPR clusters are transcribed and processed into mature CRISPR (Clustered Regularly Interspaced Short Palindromic Repeats) RNA (crRNA). Cas9 belongs to the type II CRISPR/Cas system and has strong endonuclease activity to cut target DNA.

In certain embodiments, the CRISPR/Cas-like protein can be a wild type CRISPR/Cas protein, a modified CRISPR/Cas protein, or a fragment of a wild type or modified CRISPR/Cas protein. The CRISPR/Cas-like protein can be modified to increase nucleic acid binding affinity and/or specificity, alter an enzymatic activity, and/or change another property of the protein. For example, nuclease (i.e., DNase, RNase) domains of the CRISPR/Cas-like protein can be modified, deleted, or inactivated. Alternatively, the CRISPR/Cas-like protein can be truncated to remove domains that are not essential for the function of the fusion protein. The CRISPR/Cas-like protein can also be truncated or modified to optimize the activity of the effector domain of the fusion protein.

In some embodiments, the CRISPR/Cas-like protein can be derived from a wild type Cas9 protein or fragment thereof. In other embodiments, the CRISPR/Cas-like protein can be derived from modified Cas9 protein. For example, the amino acid sequence of the Cas9 protein can be modified to alter one or more properties (e.g., nuclease activity, affinity, stability, etc.) of the protein. Alternatively, domains of the Cas9 protein not involved in RNA-guided cleavage can be eliminated from the protein such that the modified Cas9 protein is smaller than the wild type Cas9 protein.

Cas9 is guided by a mature crRNA that contains about 20 base pairs (bp) of unique target sequence (called spacer) and a trans-activated small RNA (tracrRNA) that serves as a guide for ribonuclease III-aided processing of pre-crRNA. The crRNA:tracrRNA duplex directs Cas9 to target DNA via complementary base pairing between the spacer on the crRNA and the complementary sequence (called protospacer) on the target DNA. Cas9 recognizes a trinucleotide (NGG) protospacer adjacent motif (PAM) to specify the cut site (the 3rd nucleotide from PAM). The crRNA and tracrRNA can be expressed separately or engineered into an artificial fusion small guide RNA (sgRNA) via a synthetic stem loop (AGAAAU) to mimic the natural crRNA/tracrRNA duplex. Such sgRNA, like shRNA, can be synthesized or in vitro transcribed for direct RNA transfection or expressed from U6 or H1-promoted RNA expression vector, although cleavage efficiencies of the artificial sgRNA are lower than those for systems with the crRNA and tracrRNA expressed separately. Therefore, the Cas9 gRNA technology requires the expression of the Cas9 protein and gRNA, which then form a gene editing complex at the specific target DNA binding site within the target genome and inflict cleavage/mutation of the target DNA.

However, the present disclosure is not limited to the use of Cas9-mediated gene editing. Rather, the present disclosure encompasses the use of other CRISPR-associated peptides, which can be targeted to a targeted sequence using a gRNA and can edit to target site of interest. For example, in some embodiments, the disclosure utilizes Cas12a (also known as Cpf1) to edit the target site of interest.

As described herein, CRISPR-Cas systems generally refer to an enzyme system that includes a guide RNA sequence that contains a nucleotide sequence complementary or substantially complementary to a region of a target polynucleotide, and a protein with nuclease activity. CRISPR-Cas systems include Type I CRISPR-Cas system, Type II CRISPR-Cas system, Type III CRISPR-Cas system, and derivatives thereof. CRISPR-Cas systems include engineered and/or programmed nuclease systems derived from naturally accruing CRISPR-Cas systems. In certain embodiments, CRISPR-Cas systems contain engineered and/or mutated Cas proteins. In some embodiments, nucleases generally refer to enzymes capable of cleaving the phosphodiester bonds between the nucleotide subunits of nucleic acids. In some embodiments, endonucleases are generally capable of cleaving the phosphodiester bond within a polynucleotide chain. Nickases refer to endonucleases that cleave only a single strand of a DNA duplex.

In some embodiments, the CRISPR/Cas system used herein can be a type I, a type II, or a type III system. Non-limiting examples of suitable CRISPR/Cas proteins include Cas3, Cas4, Cas5, Cas5e (or CasD), Cas6, Cas6e, Cas6f, Cas7, Cas8a1, Cas8a2, Cas8b, Cas8c, Cas9, Cas10, Cas10d, CasF, CasG, CasH, CasX, CasΦ, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cu1966. By way of further example, in some embodiments, the CRISPR-Cas protein is a Cas1, Cas1B, Cas2, Cas3, Cas4, Cas5, Cash, Cas7, Cas8, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx16, CsaX, Csx3, Csx1, Csx15, Csf1, Csf2, Csf3, Csf4, Cas9, Cas12 (e.g., Cas12a, Cas12b, Cas12c, Cas12d, Cas12k, Cas12j/CasΦ, Cas12L etc.), Cas13 (e.g., Cas13a, Cas13b (such as Cas13b-t1, Cas13b-t2, Cas13b-t3), Cas13c, Cas13d, etc.), Cas14, CasX, CasY, or an engineered form of the Cas protein. In some embodiments, the CRISPR/Cas protein or endonuclease is Cas9. In some embodiments, the CRISPR/Cas protein or endonuclease is Cas12. In certain embodiments, the Cas12 polypeptide is Cas12a, Cas12b, Cas12c, Cas12d, Cas12e, Cas12g, Cas12h, Cas12i, Cas12L or Cas12J. In some embodiments, the CRISPR/Cas protein or endonuclease is CasX. In some embodiments, the CRISPR/Cas protein or endonuclease is CasY. In some embodiments, the CRISPR/Cas protein or endonuclease is CasΦ.

In some embodiments, the Cas9 protein can be from or derived from: Staphylococcus aureus, Streptococcus pyogenes, Streptococcus thermophilus, Streptococcus sp., Nocardiopsis dassonvillei, Streptomyces pristinaespiralis, Streptomyces viridochromogenes, Streptomyces viridochromogenes, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Fine goldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, or Acaryochloris marina.

In some embodiments, the composition comprises a CRISPR-associated (Cas) protein, or functional fragment or derivative thereof. In some embodiments, the Cas protein is an endonuclease, including but not limited to the Cas9 nuclease. In some embodiments, the Cas9 protein comprises an amino acid sequence identical to the wild type Streptococcus pyogenes or Staphylococcus aureus Cas9 amino acid sequence. In some embodiments, the Cas protein comprises the amino acid sequence of a Cas protein from other species, for example other Streptococcus species, such as thermophilus; Pseudomonas aeruginosa, Escherichia co/i, or other sequenced bacteria genomes and archaea, or other prokaryotic microorganisms. Other Cas proteins, useful for the present disclosure, known or can be identified, using methods known in the art (see e.g., Esvelt et al., 2013, Nature Methods, 10: 1116-1121). In some embodiments, the Cas protein comprises a modified amino acid sequence, as compared to its natural source.

CRISPR/Cas proteins comprise at least one RNA recognition and/or RNA binding domain. RNA recognition and/or RNA binding domains interact with guide RNAs (gRNAs). CRISPR/Cas proteins can also comprise nuclease domains (i.e., DNase or RNase domains), DNA binding domains, helicase domains, RNAse domains, protein-protein interaction domains, dimerization domains, as well as other domains.

The CRISPR/Cas-like protein can be a wild type CRISPR/Cas protein, a modified CRISPR/Cas protein, or a fragment of a wild type or modified CRISPR/Cas protein. The CRISPR/Cas-like protein can be modified to increase nucleic acid binding affinity and/or specificity, alter an enzymatic activity, and/or change another property of the protein. For example, nuclease (i.e., DNase, RNase) domains of the CRISPR/Cas-like protein can be modified, deleted, or inactivated. Alternatively, the CRISPR/Cas-like protein can be truncated to remove domains that are not essential for the function of the Cas protein. The CRISPR/Cas-like protein can also be truncated or modified to optimize the activity of the effector domain of the Cas protein.

In some embodiments, the CRISPR/Cas-like protein can be derived from a wild type Cas protein or fragment thereof. In some embodiments, the CRISPR/Cas-like protein is a modified Cas9 protein. For example, the amino acid sequence of the Cas9 protein can be modified to alter one or more properties (e.g., nuclease activity, affinity, stability, etc.) of the protein relative to wild-type or another Cas protein. Alternatively, domains of the Cas9 protein not involved in RNA-guided cleavage can be eliminated from the protein such that the modified Cas9 protein is smaller than the wild-type Cas9 protein.

The disclosed CRISPR-Cas compositions should also be construed to include any form of a protein having substantial homology to a Cas protein (e.g., Cas9, saCas9, Cas9 protein) disclosed herein. In some embodiments, a protein which is “substantially homologous” is about 50% homologous, about 70% homologous, about 80% homologous, about 90% homologous, about 95% homologous, or about 99% homologous to amino acid sequence of a Cas protein disclosed herein. The Cas9 can be an orthologous. Six smaller Cas9 orthologues have been used and reports have shown that Cas9 from Staphylococcus aureus (SaCas9) can edit the genome with efficiencies similar to those of SpCas9, while being more than 1 kilobase shorter.

In some embodiments, the composition comprises a CRISPR-associated (Cas) peptide, or functional fragment or derivative thereof. In certain embodiments, the Cas peptide is an endonuclease, including but not limited to the Cas9 nuclease. In some embodiments, the Cas9 peptide comprises an amino acid sequence identical to the wild type Streptococcus pyogenes Cas9 amino acid sequence. In some embodiments, the Cas peptide may comprise the amino acid sequence of a Cas protein from other species, for example other Streptococcus species, such as thermophilus; Psuedomonas aeruginosa, Escherichia co/i, or other sequenced bacteria genomes and archaea, or other prokaryotic microogranisms. Other Cas peptides, useful for the present disclosure, known or can be identified, using methods known in the art (see e.g., Esvelt et al., 2013, Nature Methods, 10: 1116-1121). In certain embodiments, the Cas peptide may comprise a modified amino acid sequence, as compared to its natural source. For example, in some embodiments, the wild type Streptococcus pyogenes Cas9 sequence can be modified. In certain embodiments, the amino acid sequence can be codon optimized for efficient expression in human cells (i.e., “humanized) or in a species of interest. A humanized Cas9 nuclease sequence can be for example, the Cas9 nuclease sequence encoded by any of the expression vectors listed in Genbank accession numbers KM099231.1 GL669193757; KM099232.1 GL669193761; or KM099233.1 GL669193765. Alternatively, the Cas9 nuclease sequence can be for example, the sequence contained within a commercially available vector such as PX330 or PX260 from Addgene (Cambridge, MA). In some embodiments, the Cas9 endonuclease can have an amino acid sequence that is a variant or a fragment of any of the Cas9 endonuclease sequences of Genbank accession numbers KM099231.1 GL669193757; KM099232.1 GL669193761; or KM099233.1 GL669193765 or Cas9 amino acid sequence of PX330 or PX260 (Addgene, Cambridge, MA).

The Cas9 nucleotide sequence can be modified to encode biologically active variants of Cas9, and these variants can have or can include, for example, an amino acid sequence that differs from a wild type Cas9 by virtue of containing one or more mutations (e.g., an addition, deletion, or substitution mutation or a combination of such mutations). One or more of the substitution mutations can be a substitution (e.g., a conservative amino acid substitution).

In certain embodiments, the Cas peptide is a mutant Cas9, wherein the mutant Cas9 reduces the off-target effects, as compared to wild-type Cas9. In some embodiments, the mutant Cas9 is a Streptococcus pyogenes Cas9 (SpCas9) variant.

In some embodiments, SpCas9 variants comprise one or more point mutations, including, but not limited to R780A, K810A, K848A, K855A, H982A, K1003A, and R1060A (Slaymaker et al., 2016, Science, 351(6268): 84-88). In some embodiments, SpCas9 variants comprise D1135E point mutation (Kleinstiver et al., 2015, Nature, 523(7561): 481-485). In some embodiments, SpCas9 variants comprise one or more point mutations, including, but not limited to N497A, R661A, Q695A, Q926A, D1135E, L169A, and Y450A (Kleinstiver et al., 2016, Nature, doi:10.1038/nature16526). In some embodiments, SpCas9 variants comprise one or more point mutations, including but not limited to M495A, M694A, and M698A. Y450 is involved with hydrophobic base pair stacking. N497, R661, Q695, Q926 are involved with residue to base hydrogen bonding contributing to off-target effects. N497 hydrogen bonding through peptide backbone. L169A is involved with hydrophobic base pair stacking. M495A, M694A, and H698A are involved with hydrophobic base pair stacking.

In some embodiments, SpCas9 variants comprise one or more point mutations at one or more of the following residues: R780, K810, K848, K855, H982, K1003, R1060, D1135, N497, R661, Q695, Q926, L169, Y450, M495, M694, and M698. In some embodiments, SpCas9 variants comprise one or more point mutations selected from the group of: R780A, K810A, K848A, K855A, H982A, K1003A, R1060A, D1135E, N497A, R661A, Q695A, Q926A, L169A, Y450A, M495A, M694A, and M698A.

In some embodiments, the SpCas9 variant comprises the point mutations, relative to wildtype SpCas9, of N497A, R661A, Q695A, and Q926A. In some embodiments, the SpCas9 variant comprises the point mutations, relative to wildtype SpCas9, of N497A, R661A, Q695A, Q926A, and D1135E. In some embodiments, the SpCas9 variant comprises the point mutations, relative to wildtype SpCas9, of N497A, R661A, Q695A, Q926A, and L169A. In some embodiments, the SpCas9 variant comprises the point mutations, relative to wildtype SpCas9, of N497A, R661A, Q695A, Q926A, and Y450A. In some embodiments, the SpCas9 variant comprises the point mutations, relative to wildtype SpCas9, of N497A, R661A, Q695A, Q926A, and M495A. In some embodiments, the SpCas9 variant comprises the point mutations, relative to wildtype SpCas9, of N497A, R661A, Q695A, Q926A, and M694A. In some embodiments, the SpCas9 variant comprises the point mutations, relative to wildtype SpCas9, of N497A, R661A, Q695A, Q926A, and H698A. In some embodiments, the SpCas9 variant comprises the point mutations, relative to wildtype SpCas9, of N497A, R661A, Q695A, Q926A, D1135E, and L169A. In some embodiments, the SpCas9 variant comprises the point mutations, relative to wildtype SpCas9, of N497A, R661A, Q695A, Q926A, D1135E, and Y450A. In some embodiments, the SpCas9 variant comprises the point mutations, relative to wildtype SpCas9, of N497A, R661A, Q695A, Q926A, D1135E, and M495A. In some embodiments, the SpCas9 variant comprises the point mutations, relative to wildtype SpCas9, of N497A, R661A, Q695A, Q926A, D1135E, and M694A. In some embodiments, the SpCas9 variant comprises the point mutations, relative to wildtype SpCas9, of N497A, R661A, Q695A, Q926A, D1135E, and M698A.

In some embodiments, the SpCas9 variant comprises the point mutations, relative to wildtype SpCas9, of R661A, Q695A, and Q926A. In some embodiments, the SpCas9 variant comprises the point mutations, relative to wildtype SpCas9, of R661A, Q695A, Q926A, and D1135E. In some embodiments, the SpCas9 variant comprises the point mutations, relative to wildtype SpCas9, of R661A, Q695A, Q926A, and L169A. In some embodiments, the SpCas9 variant comprises the point mutations, relative to wildtype SpCas9, of R661A, Q695A, Q926A, and Y450A. In some embodiments, the SpCas9 variant comprises the point mutations, relative to wildtype SpCas9, of R661A, Q695A, Q926A, and M495A. In some embodiments, the SpCas9 variant comprises the point mutations, relative to wildtype SpCas9, of R661A, Q695A, Q926A, and M694A. In some embodiments, the SpCas9 variant comprises the point mutations, relative to wildtype SpCas9, of R661A, Q695A, Q926A, and H698A. In some embodiments, the SpCas9 variant comprises the point mutations, relative to wildtype SpCas9, of R661A, Q695A, Q926A, D1135E, and L169A. In some embodiments, the SpCas9 variant comprises the point mutations, relative to wildtype SpCas9, of R661A, Q695A, Q926A, D1135E, and Y450A. In some embodiments, the SpCas9 variant comprises the point mutations, relative to wildtype SpCas9, of R661A, Q695A, Q926A, D1135E, and M495A. In some embodiments, the SpCas9 variant comprises the point mutations, relative to wildtype SpCas9, of R661A, Q695A, Q926A, D1135E, and M694A. In some embodiments, the SpCas9 variant comprises the point mutations, relative to wildtype SpCas9, of R661A, Q695A, Q926A, D1135E, and M698A.

In some embodiments, the mutant Cas9 comprises one or more mutations that alter PAM specificity (Kleinstiver et al., 2015, Nature, 523(7561):481-485; Kleinstiver et al., 2015, Nat Biotechnol, 33(12): 1293-1298). In some embodiments, the mutant Cas9 comprises one or more mutations that alter the catalytic activity of Cas9, including but not limited to D10A in RuvC and H840A in HNH (Cong et al., 2013; Science 339: 919-823, Gasiubas et al., 2012; PNAS 109:E2579-2586 Jinek et al., 2012; Science 337: 816-821).

In addition to the wild type and variant Cas9 endonucleases described, embodiments of the disclosure also encompass CRISPR systems including newly developed “enhanced-specificity” S. pyogenes Cas9 variants (eSpCas9), which dramatically reduce off target cleavage. These variants are engineered with alanine substitutions to neutralize positively charged sites in a groove that interacts with the non-target strand of DNA. This aim of this modification is to reduce interaction of Cas9 with the non-target strand, thereby encouraging re-hybridization between target and non-target strands. The effect of this modification is a requirement for more stringent Watson-Crick pairing between the gRNA and the target DNA strand, which limits off-target cleavage (Slaymaker, I. M. et al. (2015) DOI:10.1126/science.aad5227).

In certain embodiments, three variants found to have the best cleavage efficiency and fewest off-target effects: SpCas9 (K855A), SpCas9 (K810A/K1003A/R1060A) (a.k.a. eSpCas9 1.0), and SpCas9(K848A/K1003A/R1060A) (a.k.a. eSPCas9 1.1) are employed in the compositions. The disclosure is by no means limited to these variants, and also encompasses all Cas9 variants (Slaymaker, I. M. et al. (2015)). The present disclosure also includes another type of enhanced specificity Cas9 variant, “high fidelity” spCas9 variants (HF-Cas9). Examples of high fidelity variants include SpCas9-HF1 (N497A/R661A/Q695A/Q926A), SpCas9-HF2 (N497A/R661A/Q695A/Q926A/D1135E), SpCas9-HF3 (N497A/R661A/Q695A/Q926A/L169A), SpCas9-HF4 (N497A/R661A/Q695A/Q926A/Y450A). Also included are all SpCas9 variants bearing all possible single, double, triple and quadruple combinations of N497A, R661A, Q695A, Q926A or any other substitutions (Kleinstiver, B. P. et al., 2016, Nature. DOI: 10.1038/nature16526).

Accordingly, in certain embodiments, a Cas9 variant comprises a human-optimized Cas9; a nickase mutant Cas9; saCas9; enhanced-fidelity SaCas9 (efSaCas9); SpCas9(K855a); SpCas9(K810A/K1003A/r1060A); SpCas9(K848A/K1003A/R1060A); SpCas9 N497A, R661A, Q695A, Q926A; SpCas9 N497A, R661A, Q695A, Q926A, D1135E; SpCas9 N497A, R661A, Q695A, Q926A L169A; SpCas9 N497A, R661A, Q695A, Q926A Y450A; SpCas9 N497A, R661A, Q695A, Q926A M495A; SpCas9 N497A, R661A, Q695A, Q926A M694A; SpCas9 N497A, R661A, Q695A, Q926A H698A; SpCas9 N497A, R661A, Q695A, Q926A, D1135E, L169A; SpCas9 N497A, R661A, Q695A, Q926A, D1135E, Y450A; SpCas9 N497A, R661A, Q695A, Q926A, D1135E, M495A; SpCas9 N497A, R661A, Q695A, Q926A, D1135E, M694A; SpCas9 N497A, R661A, Q695A, Q926A, D1135E, M698A; SpCas9 R661A, Q695A, Q926A; SpCas9 R661A, Q695A, Q926A, D1135E; SpCas9 R661A, Q695A, Q926A, L169A; SpCas9 R661A, Q695A, Q926A Y450A; SpCas9 R661A, Q695A, Q926A M495A; SpCas9 R661A, Q695A, Q926A M694A; SpCas9 R661A, Q695A, Q926A H698A; SpCas9 R661A, Q695A, Q926A D1135E L169A; SpCas9 R661A, Q695A, Q926A D1135E Y450A; SpCas9 R661A, Q695A, Q926A D1135E M495A; or SpCas9 R661A, Q695A, Q926A, D1135E or M694A.

As used herein, the term “Cas” is meant to include all Cas molecules comprising variants, mutants, orthologues, high-fidelity variants and the like.

However, the present disclosure is not limited to the use of Cas9-mediated gene editing. Rather, the present disclosure encompasses the use of other CRISPR-associated peptides, which can be targeted to a targeted sequence using a gRNA and can edit to target site of interest. For example, in some embodiments, the disclosure utilizes Cpf1 to edit the target site of interest. Cpf1 is a single crRNA-guided, class 2 CRISPR effector protein which can effectively edit target DNA sequences in human cells. Exemplary Cpf1 includes, but is not limited to, Acidaminiococcus sp. Cpf1 (AsCpf1) and Lachnospiraceae bacterium Cpf1 (LbCpf1).

Guide Nucleic Acid Sequences: In some embodiments, the gRNA comprises a crRNA:tracrRNA duplex. In some embodiments, the gRNA comprises a stem-loop that mimics the natural duplex between the crRNA and tracrRNA. In some embodiments, the stem-loop comprises a nucleotide sequence comprising AGAAAU. For example in some embodiments, the composition comprises a synthetic or chimeric guide RNA comprising a crRNA, stem, and tracrRNA.

In certain embodiments, the composition comprises an isolated crRNA and/or an isolated tracrRNA which hybridize to form a natural duplex. For example, in some embodiments, the gRNA comprises a crRNA or crRNA precursor (pre-crRNA) comprising a targeting sequence.

The guide RNA sequence can be a sense or anti-sense sequence. The guide RNA sequence generally includes a proto-spacer adjacent motif (PAM). The sequence of the PAM can vary depending upon the specificity requirements of the CRISPR endonuclease used. In the CRISPR-Cas system derived from S. pyogenes, the target DNA typically immediately precedes a 5′-NGG proto-spacer adjacent motif (PAM). Thus, for the S. pyogenes Cas9, the PAM sequence can be AGG, TGG, CGG or GGG. Other Cas9 orthologs may have different PAM specificities. For example, Cas9 from S. thermophilus requires 5′-NNAGAA for CRISPR 1 and 5′-NGGNG for CRISPR3) and Neiseria menigiditis requires 5′-NNNNGATT). The specific sequence of the guide RNA may vary. The length of the guide RNA sequence can vary from about 20 to about 60 or more nucleotides, for example about 20, about 21, about 22, about 23, about 24, about 25, about 26, about 27, about 28, about 29, about 30, about 31, about 32, about 33, about 34, about 35, about 36, about 37, about 38, about 39, about 40, about 45, about 50, about 55, about 60 or more nucleotides. Useful selection methods identify regions having extremely low homology between the foreign viral genome and host cellular genome including endogenous retroviral DNA, include bioinformatic screening using 12-bp+NGG target-selection criteria to exclude off-target human transcriptome or (even rarely) untranslated-genomic sites.

The guide RNA sequence can be configured as a single sequence or as a combination of one or more different sequences, e.g., a multiplex configuration. Multiplex configurations can include combinations of two, three, four, five, six, seven, eight, nine, ten, or more different guide RNAs.

When the compositions are administered in an expression vector, the guide RNAs can be encoded by a single vector. Alternatively, multiple vectors can be engineered to each include two or more different guide RNAs.

When the compositions are administered as a nucleic acid or are contained within an expression vector, the CRISPR endonuclease can be encoded by the same nucleic acid or vector as the guide RNA sequences. Alternatively, or in addition, the CRISPR endonuclease can be encoded in a physically separate nucleic acid from the guide RNA sequences or in a separate vector.

In some embodiments, the RNA molecules e.g. crRNA, tracrRNA, gRNA are engineered to comprise one or more modified nucleobases. For example, known modifications of RNA molecules can be found, for example, in Genes VI, Chapter 9 (“Interpreting the Genetic Code”), Lewis, ed. (1997, Oxford University Press, New York), and Modification and Editing of RNA, Grosjean and Benne, eds. (1998, ASM Press, Washington D.C.). Modified RNA components include the following: 2′-O-methylcytidine; N⁴-methylcytidine; N⁴-2′-O-dimethylcytidine; N⁴-acetylcytidine; 5-methylcytidine; 5,2′-O-di methylcytidine; 5-hydroxymethylcytidine; 5-formylcytidine; 2′-O-methyl-5-formaylcytidine; 3-methylcytidine; 2-thiocytidine; lysidine; 2′-O-methyluridine; 2-thiouridine; 2-thio-2′-O-methyluridine; 3,2′-O-dimethyluridine; 3-(3-amino-3-carboxypropyl)uridine; 4-thiouridine; ribosylthymine; 5,2′-O-dimethyluridine; 5-methyl-2-thiouridine; 5-hydroxyuridine; 5-methoxyuridine; uridine 5-oxyacetic acid; uridine 5-oxyacetic acid methyl ester; 5-carboxymethyluridine; 5-methoxycarbonylmethyluridine; 5-methoxycarbonylmethyl-2′-O-methyluridine; 5-methoxycarbonylmethyl-2′-thiouridine; 5-carbamoylmethyluridine; 5-carbamoylmethyl-2′-O-methyluridine; 5-(carboxyhydroxymethyl)uridine; 5-(carboxyhydroxymethyl) uridinemethyl ester; 5-aminomethyl-2-thiouridine; 5-methylaminomethyluridine; 5-methylaminomethyl-2-thiouridine; 5-methylaminomethyl-2-selenouridine; 5-carboxymethylaminomethyluridine; 5-carboxymethylaminomethyl-2′-O-methyl-uridine; 5-carboxymethylaminomethyl-2-thiouridine; dihydrouridine; dihydroribosylthymine; 2′-methyladenosine; 2-methyladenosine; N⁶N-methyladenosine; N⁶,N⁶-dimethyladenosine; N⁶,2′-O-trimethyladenosine; 2-methylthio-N⁶N-isopentenyladenosine; N⁶-(cis-hydroxyisopentenyl)-adenosine; 2-methylthio-N⁶-(cis-hydroxyisopentenyl)-adenosine; N⁶-glycinylcarbamoyl)adenosine; N⁶-threonylcarbamoyl adenosine; N⁶-methyl-N⁶-threonylcarbamoyl adenosine; 2-methylthio-N⁶-methyl-N⁶-threonylcarbamoyl adenosine; N⁶-hydroxynorvalylcarbamoyl adenosine; 2-methylthio-N⁶-hydroxnorvalylcarbamoyl adenosine; 2′-O-ribosyladenosine (phosphate); inosine; 2′O-methyl inosine; 1-methyl inosine; 1;2′-O-dimethyl inosine; 2′-O-methyl guanosine; 1-methyl guanosine; N²-methyl guanosine; N²,N²-dimethyl guanosine; N²,2′-O-dimethyl guanosine; N²,N²,2′-O-trimethyl guanosine; 2′-O-ribosyl guanosine (phosphate); 7-methyl guanosine; N²;7-dimethyl guanosine; N²; N²;7-trimethyl guanosine; wyosine; methylwyosine; under-modified hydroxywybutosine; wybutosine; hydroxywybutosine; peroxywybutosine; queuosine; epoxyqueuosine; galactosyl-queuosine; mannosyl-queuosine; 7-cyano-7-deazaguanosine; arachaeosine [also called 7-formamido-7-deazaguanosine]; and 7-aminomethyl-7-deazaguanosine.

In certain embodiments, the composition comprises multiple different gRNAs, each targeted to a different target sequence. In certain embodiments, this multiplexed strategy provides for increased efficacy. In some embodiments, the compositions described herein utilize about 1 gRNA to about 6 gRNAs. In some embodiments, the compositions described herein utilize at least about 1 gRNA. In some embodiments, the compositions described herein utilize at most about 6 gRNAs. In some embodiments, the compositions described herein utilize about 1 gRNA to about 2 gRNAs, about 1 gRNA to about 3 gRNAs, about 1 gRNA to about 4 gRNAs, about 1 gRNA to about 5 gRNAs, about 1 gRNA to about 6 gRNAs, about 2 gRNAs to about 3 gRNAs, about 2 gRNAs to about 4 gRNAs, about 2 gRNAs to about 5 gRNAs, about 2 gRNAs to about 6 gRNAs, about 3 gRNAs to about 4 gRNAs, about 3 gRNAs to about 5 gRNAs, about 3 gRNAs to about 6 gRNAs, about 4 gRNAs to about 5 gRNAs, about 4 gRNAs to about 6 gRNAs, or about 5 gRNAs to about 6 gRNAs. In some embodiments, the compositions described herein utilize about 1 gRNA, about 2 gRNAs, about 3 gRNAs, about 4 gRNAs, about 5 gRNAs, or about 6 gRNAs.

In some embodiments, the gRNA is a synthetic oligonucleotide. In some embodiments, the synthetic nucleotide comprises a modified nucleotide. Modification of the inter-nucleoside linker (i.e. backbone) can be utilized to increase stability or pharmacodynamic properties. For example, inter-nucleoside linker modifications prevent or reduce degradation by cellular nucleases, thus increasing the pharmacokinetics and bioavailability of the gRNA. Generally, a modified inter-nucleoside linker includes any linker other than other than phosphodiester (PO) liners, that covalently couples two nucleosides together. In some embodiments, the modified inter-nucleoside linker increases the nuclease resistance of the gRNA compared to a phosphodiester linker. For naturally occurring oligonucleotides, the inter-nucleoside linker includes phosphate groups creating a phosphodiester bond between adjacent nucleosides. In some embodiments, the gRNA comprises one or more inter-nucleoside linkers modified from the natural phosphodiester. In some embodiments all of the inter-nucleoside linkers of the gRNA, or contiguous nucleotide sequence thereof, are modified. For example, in some embodiments the inter-nucleoside linkage comprises sulfur (S), such as a phosphorothioate inter-nucleoside linkage.

Modifications to the ribose sugar or nucleobase can also be utilized herein. Generally, a modified nucleoside includes the introduction of one or more modifications of the sugar moiety or the nucleobase moiety. In some embodiments, the gRNAs, as described, comprise one or more nucleosides comprising a modified sugar moiety, wherein the modified sugar moiety is a modification of the sugar moiety when compared to the ribose sugar moiety found in deoxyribose nucleic acid (DNA) and RNA. Numerous nucleosides with modification of the ribose sugar moiety can be utilized, primarily with the aim of improving certain properties of oligonucleotides, such as affinity and/or stability. Such modifications include those where the ribose ring structure is modified. These modifications include replacement with a hexose ring (HNA), a bicyclic ring having a biradical bridge between the C2 and C4 carbons on the ribose ring (e.g. locked nucleic acids (LNA)), or an unlinked ribose ring which typically lacks a bond between the C2 and C3 carbons (e.g. UNA). Other sugar modified nucleosides include, for example, bicyclohexose nucleic acids or tricyclic nucleic acids. Modified nucleosides also include nucleosides where the sugar moiety is replaced with a non-sugar moiety, for example in the case of peptide nucleic acids (PNA), or morpholino nucleic acids.

Sugar modifications also include modifications made by altering the substituent groups on the ribose ring to groups other than hydrogen, or the 2′-OH group naturally found in DNA and RNA nucleosides. Substituents may, for example be introduced at the 2′, 3′, 4′ or 5′ positions. Nucleosides with modified sugar moieties also include 2′ modified nucleosides, such as 2′ substituted nucleosides. Indeed, much focus has been spent on developing 2′ substituted nucleosides, and numerous 2′ substituted nucleosides have been found to have beneficial properties when incorporated into oligonucleotides, such as enhanced nucleoside resistance and enhanced affinity. A 2′ sugar modified nucleoside is a nucleoside that has a substituent other than H or —OH at the 2′ position (2′ substituted nucleoside) or comprises a 2′ linked biradicle, and includes 2′ substituted nucleosides and LNA (2′-4′ biradicle bridged) nucleosides. Examples of 2′ substituted modified nucleosides are 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, and 2′-F-ANA nucleoside. By way of further example, in some embodiments, the modification in the ribose group comprises a modification at the 2′ position of the ribose group. In some embodiments, the modification at the 2′ position of the ribose group is selected from the group consisting of 2′-O-methyl, 2′-fluoro, 2′-deoxy, and 2′-O-(2-methoxyethyl).

In some embodiments, the gRNA comprises one or more modified sugars. In some embodiments, the gRNA comprises only modified sugars. In certain embodiments, the gRNA comprises greater than 10%, 25%, 50%, 75%, or 90% modified sugars. In some embodiments, the modified sugar is a bicyclic sugar. In some embodiments, the modified sugar comprises a 2′-O-methoxyethyl group. In some embodiments, the gRNA comprises both inter-nucleoside linker modifications and nucleoside modifications.

Target specificity can be used in reference to a guide RNA, or a crRNA specific to a target polynucleotide sequence or region and further includes a sequence of nucleotides capable of selectively annealing/hybridizing to a target (sequence or region) of a target polynucleotide (e.g. corresponding to a target), e.g., a target DNA. In some embodiments, a crRNA or the derivative thereof contains a target-specific nucleotide region complementary to a region of the target DNA sequence. In some embodiments, a crRNA or the derivative thereof contains other nucleotide sequences besides a target-specific nucleotide region. In some embodiments, the other nucleotide sequences are from a tracrRNA sequence.

gRNAs are generally supported by a scaffold, wherein a scaffold refers to the portions of gRNA or crRNA molecules comprising sequences which are substantially identical or are highly conserved across natural biological species (e.g. not conferring target specificity). Scaffolds include the tracrRNA segment and the portion of the crRNA segment other than the polynucleotide-targeting guide sequence at or near the 5′ end of the crRNA segment, excluding any unnatural portions comprising sequences not conserved in native crRNAs and tracrRNAs. In some embodiments, the crRNA or tracrRNA comprises a modified sequence. In certain embodiments, the crRNA or tracrRNA comprises at least 1, 2, 3, 4, 5, 10, or 15 modified bases (e.g. a modified native base sequence).

Complementary, as used herein, generally refers to a polynucleotide that includes a nucleotide sequence capable of selectively annealing to an identifying region of a target polynucleotide under certain conditions. As used herein, the term “substantially complementary” and grammatical equivalents is intended to mean a polynucleotide that includes a nucleotide sequence capable of specifically annealing to an identifying region of a target polynucleotide under certain conditions. Annealing refers to the nucleotide base-pairing interaction of one nucleic acid with another nucleic acid that results in the formation of a duplex, triplex, or other higher-ordered structure. The primary interaction is typically nucleotide base specific, e.g., A:T, A:U, and G:C, by Watson-Crick and Hoogsteen-type hydrogen bonding. In some embodiments, base-stacking and hydrophobic interactions can also contribute to duplex stability. Conditions under which a polynucleotide anneals to complementary or substantially complementary regions of target nucleic acids are well known in the art, e.g., as described in Nucleic Acid Hybridization, A Practical Approach, Hames and Higgins, eds., TRL Press, Washington, D.C. (1985) and Wetmur and Davidson, Mol. Biol. 31:349 (1968). Annealing conditions will depend upon the particular application and can be routinely determined by persons skilled in the art, without undue experimentation. Hybridization generally refers to process in which two single-stranded polynucleotides bind non-covalently to form a stable double-stranded polynucleotide. A resulting double-stranded polynucleotide is a “hybrid” or “duplex.” In certain instances, 100% sequence identity is not required for hybridization and, in certain embodiments, hybridization occurs at about greater than 70%, 75%, 80%, 85%, 90%, or 95% sequence identity. In certain embodiments, sequence identity includes in addition to non-identical nucleobases, sequences comprising insertions and/or deletions.

The nucleic acid of the disclosure, including the RNA (e.g., crRNA, tracrRNA, gRNA) or nucleic acids encoding the RNA, may be produced by standard techniques. For example, polymerase chain reaction (PCR) techniques can be used to obtain an isolated nucleic acid containing a nucleotide sequence described herein, including nucleotide sequences encoding a polypeptide described herein. PCR can be used to amplify specific sequences from DNA as well as RNA, including sequences from total genomic DNA or total cellular RNA. Various PCR methods are described in, for example, PCR Primer: A Laboratory Manual, 2^{nd} edition, Dieffenbach and Dveksler, eds., Cold Spring Harbor Laboratory Press, 2003. Generally, sequence information from the ends of the region of interest or beyond is employed to design oligonucleotide primers that are identical or similar in sequence to opposite strands of the template to be amplified. Various PCR strategies also are available by which site-specific nucleotide sequence modifications can be introduced into a template nucleic acid.

The isolated nucleic acids also can be chemically synthesized, either as a single nucleic acid (e.g., using automated DNA synthesis in the 3′ to 5′ direction using phosphoramidite technology) or as a series of oligonucleotides. Isolated nucleic acids of the disclosure also can be obtained by mutagenesis of, e.g., a naturally occurring portion crRNA, tracrRNA, RNA-encoding DNA, or of a Cas9-encoding DNA

In certain embodiments, the isolated RNA are synthesized from an expression vector encoding the RNA molecule.

In some embodiments, a gRNA target sequence comprises a sequence at least or about 70%, 80%, 85%, 90%, 91%, 92%, 93%, 94%, 95%, 96%, 97%, 98%, 99%, or 100% sequence identity to a targeted nucleic acid sequence within SEQ ID NO: 2. In some instances, a gRNA target sequence comprises a sequence of at least or about 95% homology to a targeted nucleic acid sequence within SEQ ID NO: 2. In some instances, a gRNA target sequence comprises a sequence at least or about 95% homology to a sequence complementary to a targeted nucleic acid sequence within SEQ ID NO: 2. In some instances, a gRNA target sequence comprises a sequence of at least or about 97% homology to a targeted nucleic acid sequence within SEQ ID NO: 2. In some instances, a gRNA target sequence comprises a sequence of at least or about 97% homology to a sequence complementary within SEQ ID NO: 2. In some instances, a gRNA target sequence comprises a sequence at least or about 99% homology to a targeted nucleic acid sequence within SEQ ID NO: 2. In some instances, a gRNA target sequence comprises a sequence at least or about 99% homology to a sequence complementary to a targeted nucleic acid sequence within SEQ ID NO: 2. In some instances, a gRNA target sequence comprises a sequence at least or about 100% homology to a targeted nucleic acid sequence within SEQ ID NO: 2. In some instances, a gRNA target sequence comprises a sequence at least or about 100% homology to a sequence complementary to a targeted nucleic acid sequence within SEQ ID NO: 2. In certain embodiments, the gRNAs are targeted to regulatory sequences.

In certain embodiments, a composition comprises a viral vector encoding a gene editing agent and at least one guide RNA (gRNA) wherein the gRNA is complementary to a target nucleic acid sequence of SEQ ID NO: 2.

Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art.

In some embodiments, lentivirus vectors are used. For example, vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity. In some embodiments, the composition includes a vector derived from an adeno-associated virus (AAV). Adeno-associated viral (AAV) vectors have become powerful gene delivery tools for the treatment of various disorders. AAV vectors possess a number of features that render them ideally suited for gene therapy, including a lack of pathogenicity, minimal immunogenicity, and the ability to transduce postmitotic cells in a stable and efficient manner. Expression of a particular gene contained within an AAV vector can be specifically targeted to one or more types of cells by choosing the appropriate combination of AAV serotype, promoter, and delivery method.

Further provided are nucleic acids encoding the CRISPR-Cas systems described herein. Provided herein are adeno-associated virus (AAV) vectors comprising nucleic acids encoding the CRISPR-Cas systems described herein. In certain instances, an AAV vector includes to any vector that comprises or derives from components of AAV and is suitable to infect mammalian cells, including human cells, of any of a number of tissue types, such as brain, heart, lung, skeletal muscle, liver, kidney, spleen, or pancreas, whether in vitro or in vivo. In certain instances, an AAV vector includes an AAV type viral particle (or virion) comprising a nucleic acid encoding a protein of interest (e.g. CRISPR-Cas systems described herein). In some embodiments, as further described herein, the AAVs disclosed herein are be derived from various serotypes, including combinations of serotypes (e.g., “pseudotyped” AAV) or from various genomes (e.g., single-stranded or self-complementary). In some embodiments, the AAV vector is a human serotype AAV vector. In such embodiments, a human serotype AAV is derived from any known serotype, e.g., from AAV1, AAV2, AAV4, AAV6, or AAV9. In some embodiments, the serotype is AAV1, AAV2, AAV3, AAV4, AAV5, AAV6, AAV7, AAV8, AAV9, AAV10, AAV11, AAVDJ, or AAVDJ/8.

In some embodiments, the composition includes a vector derived from an adeno-associated virus (AAV). AAV vectors possess a number of features that render them ideally suited for gene therapy, including a lack of pathogenicity, minimal immunogenicity, and the ability to transduce postmitotic cells in a stable and efficient manner. Expression of a particular gene contained within an AAV vector can be specifically targeted to one or more types of cells by choosing the appropriate combination of AAV serotype, promoter, and delivery method.

A variety of different AAV capsids have been described and can be used, although AAV which preferentially target the liver and/or deliver genes with high efficiency are particularly desired. The sequences of the AAV8 are available from a variety of databases. While the examples utilize AAV vectors having the same capsid, the capsid of the gene editing vector and the AAV targeting vector are the same AAV capsid. Another suitable AAV is, e.g., rh10 (WO 2003/042397). Still other AAV sources include, e.g., AAV9 (see, for example, U.S. Pat. No. 7,906,111; US 2011-0236353-A1), and/or hu37 (see, e.g., U.S. Pat. No. 7,906,111; US 2011-0236353-A1), AAV1, AAV2, AAV3, AAV4, AAV5, AAV6, AAV6.2, AAV7, AAV8, (U.S. Pat. Nos. 7,790,449; 7,282,199, WO 2003/042397; WO 2005/033321, WO 2006/110689; U.S. Pat. Nos. 7,790,449; 7,282,199; 7,588,772). Still other AAV can be selected, optionally taking into consideration tissue preferences of the selected AAV capsid.

In some embodiments, AAV vectors disclosed herein include a nucleic acid encoding a CRISPR-Cas systems described herein. In some embodiments, the nucleic acid also includes one or more regulatory sequences allowing expression and, in some embodiments, secretion of the protein of interest, such as e.g., a promoter, enhancer, polyadenylation signal, an internal ribosome entry site (“IRES”), a sequence encoding a protein transduction domain (“PTD”), and the like. Thus, in some embodiments, the nucleic acid comprises a promoter region operably linked to the coding sequence to cause or improve expression of the protein of interest in infected cells. Such a promoter can be ubiquitous, cell- or tissue-specific, strong, weak, regulated, chimeric, etc., for example, to allow efficient and stable production of the protein in the infected tissue. In certain embodiments, the promoter is homologous to the encoded protein, or heterologous, although generally promoters of use in the disclosed methods are functional in human cells. Examples of regulated promoters include, without limitation, Tet on/off element-containing promoters, rapamycin-inducible promoters, tamoxifen-inducible promoters, and metallothionein promoters. In certain embodiments. other promoters used include promoters that are tissue specific for tissues such as kidney, spleen, and pancreas. Examples of ubiquitous promoters include viral promoters, particularly the CMV promoter, the RSV promoter, the SV40 promoter, etc., and cellular promoters such as the phosphoglycerate kinase (PGK) promoter and the b-actin promoter.

In some embodiments, the recombinant AAV vector comprises packaged within an AAV capsid, a nucleic acid, generally containing a 5′ AAV ITR, the expression cassettes described herein and a 3′ AAV ITR. As described herein, in some embodiments, an expression cassette contains regulatory elements for an open reading frame(s) within each expression cassette and the nucleic acid optionally contains additional regulatory elements. The AAV vector, in some embodiments, comprises a full-length AAV 5′ inverted terminal repeat (ITR) and a full-length 3′ ITR. A shortened version of the 5′ ITR, termed ΔITR, has been described in which the D-sequence and terminal resolution site (trs) are deleted. The abbreviation “sc” refers to self-complementary. “Self-complementary AAV” refers a construct in which a coding region carried by a recombinant AAV nucleic acid sequence has been designed to form an intra-molecular double-stranded DNA template. Upon infection, rather than waiting for cell mediated synthesis of the second strand, the two complementary halves of scAAV will associate to form one double stranded DNA (dsDNA) unit that is ready for immediate replication and transcription (see, for example, D M McCarty et al, “Self-complementary recombinant adeno-associated virus (scAAV) vectors promote efficient transduction independently of DNA synthesis”, Gene Therapy, (August 2001); see also, for example, U.S. Pat. Nos. 6,596,535; 7,125,717; and 7,456,683). Where a pseudotyped AAV is to be produced, the ITRs are selected from a source which differs from the AAV source of the capsid. For example, in some embodiments, AAV2 ITRs are selected for use with an AAV capsid having a particular efficiency for a selected cellular receptor, target tissue or viral target. In some embodiments, the ITR sequences from AAV2, or the deleted version thereof (ΔITR), are used for convenience and to accelerate regulatory approval (i.e. pseudotyped). In some embodiments, a single-stranded AAV viral vector is used.

Methods for generating and isolating AAV viral vectors suitable for delivery to a subject are known in the art (see, for example, U.S. Pat. Nos. 7,790,449; 7,282,199; WO 2003/042397; WO 2005/033321, WO 2006/110689; and U.S. Pat. No. 7,588,772 B2, U.S. Pat. Nos. 5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065). In one system, a producer cell line is transiently transfected with a construct that encodes the transgene flanked by ITRs and a construct(s) that encodes rep and cap. In a second system, a packaging cell line that stably supplies rep and cap is transfected (transiently or stably) with a construct encoding the transgene flanked by ITRs. In each of these systems, AAV virions are produced in response to infection with helper adenovirus or herpesvirus, requiring the separation of the rAAVs from contaminating virus. More recently, systems have been developed that do not require infection with helper virus to recover the AAV—the required helper functions (i.e., adenovirus E1, E2a, VA, and E4 or herpesvirus UL5, UL8, UL52, and UL29, and herpesvirus polymerase) are also supplied, in trans, by the system. In these newer systems, the helper functions can be supplied by transient transfection of the cells with constructs that encode the required helper functions, or the cells can be engineered to stably contain genes encoding the helper functions, the expression of which can be controlled at the transcriptional or posttranscriptional level. In yet another system, the transgene flanked by ITRs and rep/cap genes are introduced into insect cells by infection with baculovirus-based vectors.

The CRISPR-Cas systems, for instance a Cas9, and/or any of the present RNAs, for instance a guide RNA, can be delivered using adeno associated virus (AAV), lentivirus, adenovirus or other viral vector types, or combinations thereof. Cas9 and one or more guide RNAs can be packaged into one or more viral vectors. In some embodiments, the viral vector is delivered to the tissue of interest by, for example, an intramuscular injection, while other times the viral delivery is via intravenous, transdermal, intranasal, oral, mucosal, or other delivery methods. Such delivery can be either via a single dose, or multiple doses. One skilled in the art understands that the actual dosage to be delivered herein can vary greatly depending upon a variety of factors, such as the vector chose, the target cell, organism, or tissue, the general condition of the subject to be treated, the degree of transformation/modification sought, the administration route, the administration mode, the type of transformation/modification sought, etc.

### Methods of Treatment

The present disclosure provides methods for the treatment of vasopathy (e.g., vEDS) or connective tissue disorders in a subject in need thereof by administering to the subject a therapeutically effective amount of an agent, wherein the agent modulates endothelin-1 expression or binding to the endothelin type A and/or endothelin type B receptors (EDNRA/B), thereby treating the connective tissue disorder. The agent may be e.g. an antibody or fragment thereof, a polypeptide, a small molecule, a nucleic acid molecule, or any combination, to be used in the preparation of a medicament useful for the treatment of vasopathy (e.g., vEDS) or connective tissue disorders.

Small molecule agents for use in the present therapeutic methods may be identified and chemically synthesized using known methodology. Small molecules are usually less than about 2000 Daltons in size or alternatively up to or less than about 1500, 750, 500, 250 or 200 Daltons in size, where such small molecules are capable of providing a result in vitro or hi vivo (including in vivo models) as disclosed herein. Small molecules may be, for example, fused ring systems (including those that contain 2, 3 or more fused rings, and one or more N, O or S ring atoms such as bosentan discussed herein) and contain one or more other functional groups such as amines (primary or more preferably secondary or tertiary alkylamine moieties), aldehydes, ketones, epoxides, or alcohols. Preferred small molecule agents include EDNR antagonists, such as selective ETA receptor antagonists which affect endothelin A receptors or endothelin B receptors, or dual antagonists, which affect both endothelin A and B receptors. Exemplary EDNR antagonists include sitaxentan, ambrisentan, atrasentan, BQ-123, zibotentan, edonentan, bosentan, macitentan, and tezosentan. One preferred small molecule agent is bosentan. %

The small molecule chemical compound may be a component of a combinatorial chemical library. In this regard, it is noted that techniques for screening small molecule libraries for molecules that are capable of binding to a polypeptide target are well known in the art (see, e.g., PCT Publication Nos. WO00/00823 and WO00/39585). Combinatorial chemical libraries are a collection of multiple species of chemical compounds comprised of smaller subunits or monomers. Combinatorial libraries come in a variety of sizes, ranging from a few hundred to many hundreds of thousand different species of chemical compounds. There are also a variety of library types, including oligomeric and polymeric libraries comprised of compounds such as carbohydrates, oligonucleotides, and small organic molecules, etc. Such libraries have a variety of uses, such as immobilization and chromatographic separation of chemical compounds, as well as uses for identifying and characterizing ligands capable of binding an acceptor molecule or mediating a biological activity of interest. Various techniques for synthesizing libraries of compounds on solid-phase supports are known in the art. Solid-phase supports are typically polymeric objects with surfaces that are functionalized to bind with subunits or monomers to form the compounds of the library. Synthesis of one library typically involves a large number of solid-phase supports. To make a combinatorial library, solid-phase supports are reacted with one or more subunits of the compounds and with one or more numbers of reagents in a carefully controlled, predetermined sequence of chemical reactions That is, the library subunits are “grown” on the solid-phase supports. The larger the library, the greater the number of reactions required, complicating the task of keeping track of the chemical composition of the multiple species of compounds that make tip the library. In some embodiments, the small molecules are less than about 2000 Daltons in size, alternatively less than about 1500, 750, 500, 250 or 200 Daltons in size. %

As discussed above, suitable endothelin receptor antagonist, including small molecule endothlian receptor agonists for use in the present methods and compostions are disclosed herein and can be readily identified, including by assays such as disclosed in U.S. Pat. No. 5,334,598 where the candidate suitable endothelin receptor antagonist compound suitably exhibits an IC₅₀ or ED₅₀ of a desired threshold value such 10⁻³ or lower or 10⁻⁴ or lower in standard in vitro assays that assess endothelin receptor antagonist activity such as disclosed in U.S. Pat. No. 5,334,598.

The present disclosure also provides methods comprising combination therapy, including methods comprising combination therapy for the treatment of vasopathy (e.g., vEDS) or connective tissue disorders. As used herein, “combination therapy” or “co-therapy” includes the administration of a therapeutically effective amount of an agent described above, with at least one additional active agent, also referred to herein as an “active pharmaceutical ingredient” (“API”), as part of a treatment regimen intended to provide a beneficial effect from the co-action of the agent and the additional active agent (API, e.g., an agonist, antagonist or inhibitor). In accordance with the embodiments described below, “the additional API” is understood to refer to the at least one additional API (e.g., an agent that decreases the activity or expression of MEK, ERK, PKC and/or collagen type III alpha 1 chain) administered in a combination therapy regimen with an agent described above, i.e. an endothelin receptor antagonist such as bosentan, or other agent such as sitaxentan, ambrisentan, macitentan and/or tezosentan. In addition, it is understood that more than one of the additional APIs described below may be utilized in the regimen.

The beneficial effect of the combination includes, but is not limited to, pharmacokinetic or pharmacodynamic co-action resulting from the combination of therapeutic compounds. The beneficial effect of the combination may also relate to the mitigation of a toxicity, side effect, or adverse event associated with another agent in the combination. “Combination therapy” may be, but generally is not, intended to encompass the administration of two or more of these therapeutic compounds as part of separate monotherapy regimens that incidentally and arbitrarily result in the combinations of the present disclosure. The terms “combination therapy” or “combination therapy regimen” are not intended to encompass the administration of two or more therapeutic compounds as part of separate monotherapy regimens that incidentally and arbitrarily result in a beneficial effect that was not intended or predicted. Preferably, the administration of a composition comprising an agent which modulates endothelin-1 expression or binding to EDRNA, RDNRB, or both in combination with one or more additional APIs (e.g., an agent that decreases the activity or expression of MEK, ERK, PKC, PLC, IP3 and/or collagen type III alpha 1 chain) provides a synergistic response in the subject being treated. In this context, the term “synergistic” refers to the efficacy of the combination being more effective than the additive effects of either single therapy alone.

Accordingly, in certain embodiments, a subject in need thereof is administered one or more additional APIs that inhibit the expression or activity of mitogen activated protein kinase/extracellular signal regulated kinase (MEK), extracellular signal regulated kinase (ERK), phospholipase C (PLC), inositol triphosphate (IP3), protein kinase C (pKC) or MEK, ERK, PKC, PLC, IP3 and/or collagen type III alpha 1 chain), and thereby inhibiting the activity of ERK, PLC, IP3, or PKC. In certain embodiments, a subject in need thereof is administered one or more agents that inhibit the activity or expression of one or more molecules associated with the mitogen-activated protein kinase (MAPK) pathway, e.g. RAS-RAF/MEK/Extracellular signal-regulated kinase (ERK) protein kinases. Exemplary agents suitable for use as the additional API described herein are disclosed in WO 2020/081741 and WO 2022/051685, incorporated herein by reference.

In the context of combination therapy, administration of an agent (e.g. endothelin receptor antagonist) may be simultaneous with or sequential to the administration of the one or more API. In another aspect, administration of the different components of a combination therapy may be at different frequencies. The one or more additional agents may be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a compound of the present disclosure.

The one or more additional API can be formulated for co-administration with an agent of the present disclosure in a single dosage form, as described in greater detail herein. The one or more additional API can be administered separately from the dosage form that comprises the compound of the present disclosure. When the additional API is administered separately from a compound of the present disclosure, it can be by the same or a different route of administration as the compound of the instant disclosure.

Preferably, the administration of a composition comprising an agent of the present disclosure in combination with one or more additional API provides a synergistic response in the subject having a disorder, disease or condition of the present disclosure. In this context, the term “synergistic” refers to the efficacy of the combination being more effective than the additive effects of either single therapy alone. The synergistic effect of combination therapy according to the disclosure can permit the use of lower dosages and/or less frequent administration of at least one agent in the combination compared to its dose and/or frequency outside of the combination. The synergistic effect can be manifested in the avoidance or reduction of adverse or unwanted side effects associated with the use of either therapy in the combination alone.

“Combination therapy” also embraces the administration of the compounds of the present disclosure in further combination with non-drug therapies (e.g., surgery or radiation treatment). Where the combination therapy further comprises a non-drug treatment, the non-drug treatment may be conducted at any suitable time so long as a beneficial effect from the co-action of the combination of the therapeutic compounds and non-drug treatment is achieved. For example, in appropriate cases, the beneficial effect is still achieved when the non-drug treatment is temporally removed from the administration of the therapeutic compounds, perhaps by days or even weeks.

In embodiments of the methods described herein, agents may be administered alone or in combination with at least one additional API (e.g., an agent that decreases the activity or expression of MEK, ERK, PKC, PLC, IP3 and/or collagen type III alpha 1 chain) in a method for treating vasopathy (e.g., vEDS) or connective tissue disorders. In embodiments, the agent, and the at least one additional agent are administered in a single dosage form. In another aspect, the agent and the at least one additional API are administered in separate dosage forms. In embodiments, the at least one additional API is a therapeutic agent. In embodiments, the therapeutic agent is indicated for the treatment of vasopathy (e.g., vEDS) or connective tissue disorders. In another aspect, the agent is administered in combination with at least one additional API that is not for the treatment of vasopathy (e.g., vEDS) or connective tissue disorders, e.g., a second agent that serves to mitigate a toxicity or adverse event associated with another active agent being administered in the combination therapy.

In embodiments, the at least one additional API is directed towards targeted therapy, wherein the treatment targets vasopathy (e.g., vEDS) or connective tissue disorders, proteins, or the tissue environment that contributes to vasopathy (e.g., vEDS) or connective tissue disorder progression.

The term “therapeutically effective amount” refers to an amount sufficient to treat, ameliorate a symptom of, reduce the severity of, or reduce the duration of the disease, disorder or condition, or enhance or improve the therapeutic effect of another therapy, or to prevent an identified disease, disorder or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration.

An effective amount of the agent can be administered once daily, from two to five times daily, up to two times or up to three times daily, or up to eight times daily. In embodiments, the agent is administered thrice daily, twice daily, once daily, fourteen days on (four times daily, thrice daily or twice daily, or once daily) and 7 days off in a 3-week cycle, up to five or seven days on (four times daily, thrice daily or twice daily, or once daily) and 14-16 days off in 3 week cycle, or once every two days, or once a week, or once every 2 weeks, or once every 3 weeks.

An effective amount of an agent according to this invention can range from about 0.001 mg/kg to about 1000 mg/kg, from about 0.01 mg/kg to about 100 mg/kg, from about 0.1 mg/kg to about 10 mg/kg; or any range in which the low end of the range is any amount from 0.001 mg/kg and 900 mg/kg and the upper end of the range is any amount from 0.1 mg/kg and 1000 mg/kg (e.g., 0.005 mg/kg and 200 mg/kg, 0.5 mg/kg and 20 mg/kg). Effective doses will also vary, as recognized by those skilled in the art, depending on the diseases treated, route of administration, excipient usage, and the possibility of co-usage with other therapeutic treatments such as use of other agents.

In more specific aspects, an agent of the disclosure is administered at a dosage regimen of 30-300 mg/day (e.g., 30, 35, 40, 45, 50, 55, 60, 65, 70, 75, 80, 85, 90, 95, 100, 125, 150, 175, 200, 225, 250, 275, or 300 mg/day) for at least 1 week (e.g., 1, 2, 3, 4, 5, 6, 7, 8, 9, 10, 11, 12, 36, 48, or more weeks). In certain embodiments, a compound(s) embodied herein is administered at a dosage regimen of 100-300 mg/day for 4 or 16 weeks. Alternatively or subsequently, an agent embodied herein is administered at a dosage regimen of 100 mg twice a day for 8 weeks, or optionally, for 52 weeks.

### Pharmaceutical Compositions

In certain embodiments, the present invention provides for a pharmaceutical composition comprising an agent (e.g., an agent that decreases the activity or expression of MEK, PKC, PLC, IP3, collagen type III alpha 1 chain or ERK inhibitors) are employed in the present invention. The agent can be suitably formulated and introduced into a subject or the environment of a cell by any means recognized for such delivery.

A “pharmaceutical composition” is a formulation containing the agents described herein in a pharmaceutically acceptable form suitable for administration to a subject. As used herein, the phrase “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Such compositions typically include the agent and a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

As used herein, the term “pharmaceutically acceptable salt,” is a salt formed from, for example, an acid and a basic group of an agent described herein. Illustrative salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, besylate, gentisinate, fumarate, gluconate, glucaronate, saccharate, formate, benzoate, glutamate, methanesulfonate, ethanesulfonate, benzenesulfonate, p-toluenesulfonate, and pamoate (e.g., 1,1′-methylene-bis-(2-hydroxy-3-naphthoate)) salts.

A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in a selected solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

The compositions of the invention could also be formulated as nanoparticle formulations. The compounds of the invention can be administered for immediate-release, delayed-release, modified-release, sustained-release, pulsed-release and/or controlled-release applications. The pharmaceutical compositions of the invention may contain from 0.01 to 99% weight—per volume of the active material. For administration by inhalation, the compounds are delivered in the form of an aerosol spray from pressured container or dispenser which contains a suitable propellant, e.g., a gas such as carbon dioxide, or a nebulizer. Such methods include those described in U.S. Pat. No. 6,468,798.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art. The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

Toxicity and therapeutic efficacy of such compounds can be determined by standard pharmaceutical procedures in cell cultures or experimental animals, e.g., for determining the LD50 (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For a compound used in a method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of an agent (i.e., an effective dosage) depends on the agent selected. For instance, single dose amounts of an agent in the range of approximately 1 pg to 1000 mg may be administered; in some embodiments, 10, 30, 100, or 1000 pg, or 10, 30, 100, or 1000 ng, or 10, 30, 100, or 1000 pg, or 10, 30, 100, or 1000 mg may be administered. In some embodiments, 1-5 g of the compositions can be administered.

A therapeutically effective amount of the compound of the present invention can be determined by methods known in the art. In addition to depending on the agent and selected/pharmaceutical formulation used, the therapeutically effective quantities of a pharmaceutical composition of the invention will depend on the age and on the general physiological condition of the patient and the route of administration. In certain embodiments, the therapeutic doses will generally be from about 10 and 2000 mg/day and preferably from about 30 and 1500 mg/day. Other ranges may be used, including, for example, 50-500 mg/day, 50-300 mg/day, 100-200 mg/day.

Administration may be once a day, twice a day, or more often, and may be decreased during a maintenance phase of the disease or disorder, e.g. once every second or third day instead of every day or twice a day. The dose and the administration frequency will depend on the clinical signs, which confirm maintenance of the remission phase, with the reduction or absence of at least one or more preferably more than one clinical signs of the acute phase known to the person skilled in the art. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of an agent can include a single treatment or, optionally, can include a series of treatments.

It can be appreciated that the method of introducing an agent into the environment of a cell will depend on the type of cell and the makeup of its environment. Suitable amounts of an agent must be introduced and these amounts can be empirically determined using standard methods. Exemplary effective concentrations of an individual agent in the environment of a cell can be 500 millimolar or less, 50 millimolar or less, 10 millimolar or less, 1 millimolar or less, 500 nanomolar or less, 50 nanomolar or less, 10 nanomolar or less, or even compositions in which concentrations of 1 nanomolar or less can be used.

The pharmaceutical compositions can be included in a kit, container, pack, or dispenser together with instructions for administration.

### EXAMPLES

The following examples are put forth so as to provide those of ordinary skill in the art with a complete disclosure and description of how to make and use the assay, screening, and therapeutic methods of the invention, and are not intended to limit the scope of what the inventors regard as their invention.

### Example 1: Endothelin-1 Signaling Contributes to Vascular Rupture Risk in a Mouse Model of Vascular Ehlers Danlos Syndrome

### Methods

### Mice

All mice were maintained on a C57BL/6J background (#000664, The Jackson Laboratory). ET1^{fl/fl} f mice were obtained from RIKEN (RBRC06322). Tie2-Cre mice and Sm22-Cre mice were obtained from the Jackson Laboratory (#004128. #017491). All mice were genotyped according to published protocols.

Restriction enzymes were used to detect the presence or absence of the Col3a1 mutation as previously described. The G938D mutation leads to the gain of a BamHI cut site (R3136S, New England Biolabs). All mice found dead were assessed for cause of death by necropsy, noting in particular hemothorax and hemoperitoneum.

### Histology and Immunofluorescence

Mice were euthanized by isoflurane inhalation and the left common iliac artery was transected to allow for drainage. PBS (pH 7.4) and PBS containing 4% paraformaldehyde (PFA) was flushed through the left ventricle. The heart and thoracic aorta were removed en bloc and fixed in 4% PFA overnight at 4° C. Aortas were submitted for paraffin fixation and longitudinal sections 5 micrometers thick were mounted on glass slides and stained with hematoxylin & eosin (HE), Verhoeff-van Giesen (VVG), Masson's Trichrome, or Picrosirius red (PSR). Slides were imaged at 20× and 40× magnification using a Nikon Eclipse E400 microscope. Collagen content was determined by polarized PSR intensity (31) and elastin breaks were counted by a researcher blinded to genotype and treatment arm using only VVG stained sections where elastin breaks were clearly visualized.

For immunofluorescence, slides were incubated in antigen retrieval solution for 1 minute in a pressure cooker. Sections were incubated with 1% BSA for one hour at room temperature. Primary antibodies were diluted at 1:200 in 1% BSA and incubated overnight at 4° C. Three consecutive washes were performed prior to incubation with anti-rabbit secondary antibodies conjugated to Alexa Fluor 488 (Invitrogen A-11039) or anti-mouse secondary antibodies conjugated to Alexa Fluor 594 (Invitrogen R37119) at 1:200 for 1 hour at room temperature. Slides were again washed three times prior to mounting with VECTASHIELD Hard Set Mounting Media with DAPI (H-1500). The following primary antibodies were used: anti-phospho ERK1/2 (Cell Signaling Technology, 4370), anti-PKCβ (phospho S660) (Abcam, 75837), anti-Endothelin-1 (TR.ET.48.5) (Thermo Fisher Scientific, MA3005). Images were acquired on a Zeiss 780-FCS confocal microscope at ×20 magnification and are presented as maximal intensity projection.

### RNAseq

RNA was isolated from the proximal descending thoracic aorta of three mice for each condition, flushed in PBS, and directly stored into TRIzol (Invitrogen). RNA was extracted according to manufacturer's instructions and purified using the PureLink RNA Mini Kit (Invitrogen). Library prep was performed using TruSeq Stranded Total RNA with Ribo-Zero (Illumina). Sequencing was run on an Illumina HiSeq2500 using standard protocols.

### Bioinformatics

Illumina's CASAVA 1.8.4 was used to convert BCL files to FASTQ files. Default parameters were used. rsem-1.3.0 was used for running the alignments as well as generating gene and transcript expression levels. The data was aligned to “mm10” reference genome. EBseq was used for Differential Expression analysis and default parameters were used (32).

The networks and upstream regulator analyses were generated through the use of IPA (QIAGEN Inc., qiagenbioinformatics.com/products/ingenuity-pathway-analysis).

### Western Blot

Descending thoracic aortas (distal to the left subclavian branch and proximal to the diaphragm) from mice that did not die from aortic rupture and did not have any overt pathology at the time of planned sacrifice (at 2 months of age for all samples unless otherwise stated) were harvested, snap frozen in liquid nitrogen, and stored at −80° C. until processed. Protein was extracted using an automatic bead homogenizer in conjunction with a Protein Extraction Kit (Full Moon Biosystems). All protein lysis buffers contained both PhosSTOP and cOmplete™, Mini, EDTA-free Protease Inhibitor Cocktail (Roche). Western blotting was performed using LI-COR buffer and species appropriate secondary antibodies conjugated to IR-dye700 or IRdye-800 (LI-COR Biosciences), according to the manufacturer's guidelines and analyzed using LI-COR Odyssey. The following primary antibodies were used: anti-β-Actin (8H10D10) (Cell Signaling Technology, 3700), anti-phospho ERK1/2 (Cell Signaling Technology, 4370), anti-PKCβ (phospho S660) (Abcam, 75837), anti-Endothelin-1 (TR.ET.48.5) (Thermo Fisher Scientific, MA3005), anti-Endothelin-1 (TR.ET.48.5) (Abcam, 2786).

pERK and pPKC amounts were normalized to j-actin as opposed to total ERK or total PKC for a variety of practical reasons. First, it is the amount of phosphorylated protein, and not the ratio of phosphorylated to unphosphorylated protein, that drives cellular responses. Second, we are unaware of any pathophysiologic context where the amount of unphosphorylated protein is limiting (i.e. always in excess). Third, we and others have shown that the heterogeneous nature of cells that are accumulated, recruited, and/or expanded in vascular lesions generates extreme variability and potential artifact when phosphorylated to unphosphorylated ratios are used (33, 34). This is true because many of the resident and recruited cell types in the diseased aorta express high levels of the unphosphorylated protein but are not involved in the pathologic process leading to protein activation (e.g. inflammatory cells, adventitial fibroblasts). The abundance of these cells can lead to masking of the activation signal that is only present in a small subset of critical cells.

### Delivery of Medication

For drug trials in the Col3a1^{G938D/+} mice, mice were initiated on medication at weaning and continued until 2 months of age. Cobimetinib (GDC-0973/RO551404, Active Biochem) was dissolved in drinking water and filtered to reach a final concentration of 0.02 g/L giving an estimated dose of 2 mg/kg/day. Ruboxistaurin (LY333531 HCl, Selleck Chemicals) was mixed with powdered food (LabDiet) to give a concentration of 0.1 mg/g giving an estimated dose of 8 mg/kg/day. Bosentan (K10795, Advanced ChemBlocks) was mixed with powdered food (LabDiet) to give a concentration of 1.25 mg/g, giving an estimated dose of 100 mg/kg/day.

### Endothelin-1 and Nitric Oxide Quantification

Mice were euthanized by isoflurane inhalation and blood was immediately collected via cardiac puncture. Blood allowed to clot for up to 2 hours at room temperature and was spun at 2000×g for 20 minutes at 4 degrees C. and supernatant (serum) was collected. Serum was used for quantification of ET1 (Quantikine ELISA endothelin-1, R&D Systems DET100) or for quantification of nitrate (Total Nitric Oxide and Nitrate/Nitrite Parameter Assay Kit, R&D Systems KGE001) according to manufacturer's instructions.

### Statistics

All data points are presented for quantitative data, with an overlay of the mean with SEM. All statistical analysis was performed using GraphPad Prism 8 unless otherwise noted.

For data that did not pass Shapiro-Wilk normality tests, Kruskal-Wallis (nonparametric) tests were performed to evaluate significance between groups using Dunn's multiple comparison test with a p-value of <0.05 considered statistically significant. For data that did pass normality, two-way or one-way ANOVA was used with multiple comparisons, as noted in each legend.

For single comparisons, if the Shapiro-Wilk normality test was passed, then two-tailed unpaired t-tests were performed. If Shapiro-Wilk normality test did not pass, then Mann-Whitney nonparametric tests were performed.

Kaplan-Meier survival curves were compared using a log-rank (Mantel-Cox) test. Mice were censored only if unrelated to the outcome, such as for planned biochemical or histologic analysis or if the authors were directed to euthanize them by animal care staff, for malocclusion, fight wounds, or genital prolapse.

By design, each treatment trial included contemporaneous control (untreated) mice. The performance of untreated mice remained constant for the full duration of this study, allowing pooling of controls to improve statistical power, as per our usual practice (35). All findings from our drug trials are based on analyses using a universal control group with N=93 across all drug tests that started at P21. All p-values calculated by log-rank comparisons (using Prism) are reported as unadjusted p-values, given the interdependence of many of the drug mechanisms.

For all treatment trials, untreated mice were followed for the same duration—i.e. if a treatment trial did not initiate until age 21 days, control cohorts were followed starting at age 21 days.

### Study Approval

All mice were cared for under strict adherence to the Animal Care and Use Committee of the Johns Hopkins University School of Medicine.

### Results

### Endothelin-1 is Increased in the Aortic Wall of Col3a1^{G938D} Mice

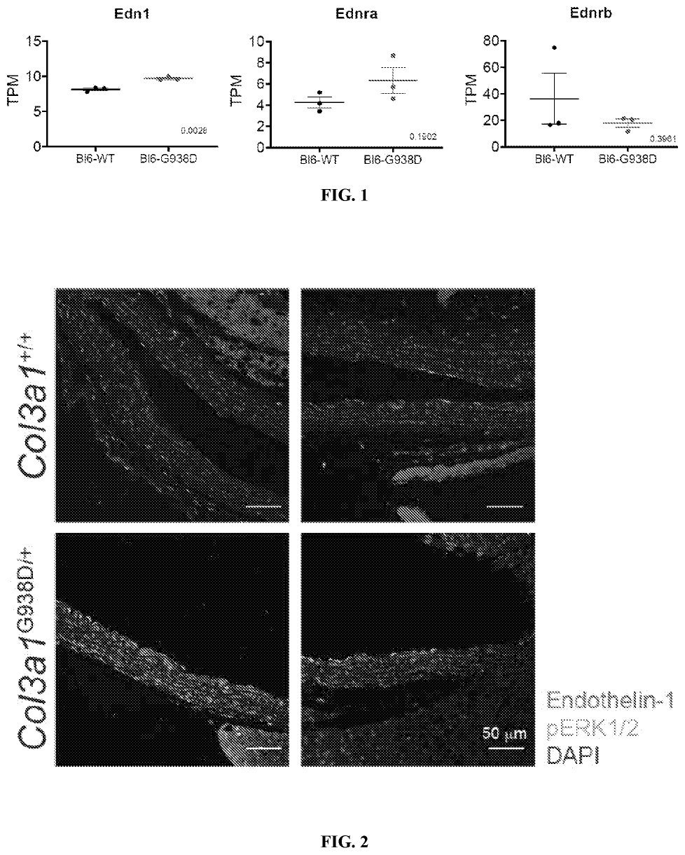

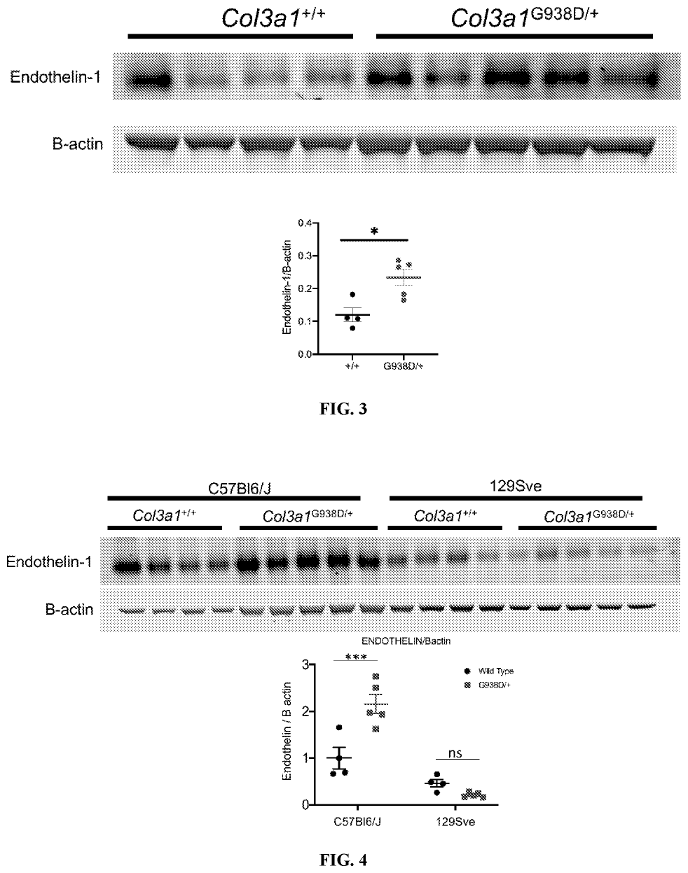

Given that angiotensin receptor blockade did not affect the risk of aortic rupture in Col3a1^{G938D} mice, a search was conducted for specific GPCR that is activated in Col3a1^{G938D} mice. The focus was on endothelin-1 (ET1), which signals through endothelin type A and endothelin type B receptors (EDNRA/B), due to its role in vascular development and disease (1-5). A small but statistically significant increase was found in the transcript of ET1, but not EDNRA/B by RNAseq (FIG. 1). Furthermore, increased amounts of endothelin-1 were detected by immunostaining of the descending thoracic aorta of Col3a1^{G938D} mice compared to Col3a1^{+/+} mice, particularly in the media of the aortic wall, primarily composed of vSMCs (FIG. 2). Furthermore, increased amounts of ET1 were detected by western blot of the descending thoracic aorta of Col3a1^{G938D} mice compared to Col3a1^{+/+} mice (FIG. 3). Informatively, in 129Sve background mice, which do not die from vascular rupture despite the Col3a1^{G938D} mutation (6), there is no increase in ET1, providing evidence that ET1 is involved in the abnormal cellular response to altered matrix (FIG. 4).

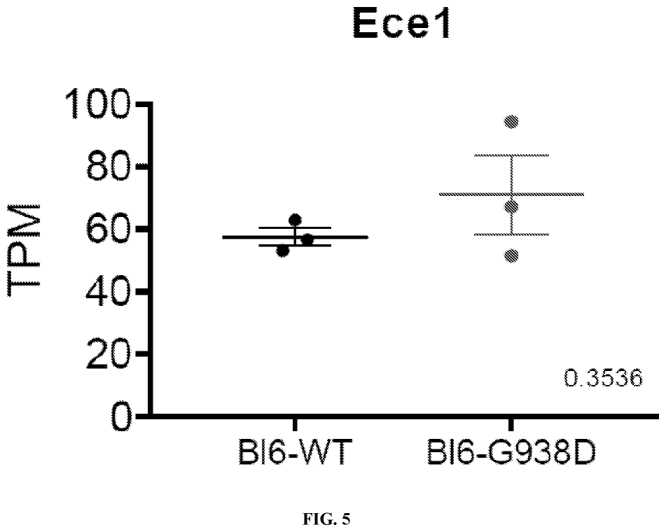

Elevations in ET1 were seen at both protein and the RNA level, however post-transcriptional or post-translational ET1 regulation may also be involved. ET1 is secreted as a proprotein and activated by endothelin converting enzyme (ECE1). ECE1 is expressed on the cell surface of vSMCs and, in disease states, increases in ECE1 activity correlate with increases in ET1 expression in the vascular media (5, 7-9), similar to what was seen by immunohistochemistry in Col3a1^{G938D} mice. There was a small but not statistically significant increase in ECE1 expression by RNAseq (FIG. 5).

### Bosentan Decreases the Risk of Dissection in Col3a1^{G938D} Mice

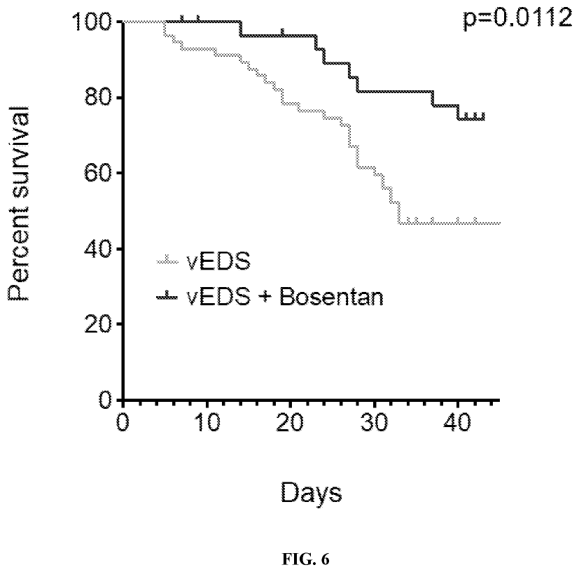

To examine the possible cause-effect relationship of the ET1 pathway and aortic rupture risk in Col3a1^{G938D} mice, pharmacological experiments were performed with bosentan, an endothelin receptor (EDNRA/B) antagonist(10). The drug was administered starting at weaning age (P21) and mice were followed for survival for 45 days. Treatment with bosentan moderately but significantly improved survival in vEDS mice, with 70% of mice surviving to the end of the trial 45 days later, compared to 46% of untreated vEDS mice, providing evidence that abnormal endothelin receptor activation contributes to vascular rupture risk (FIG. 6).

Elevated p-PKC but not p-ERK1/2 is Reversed by Bosentan

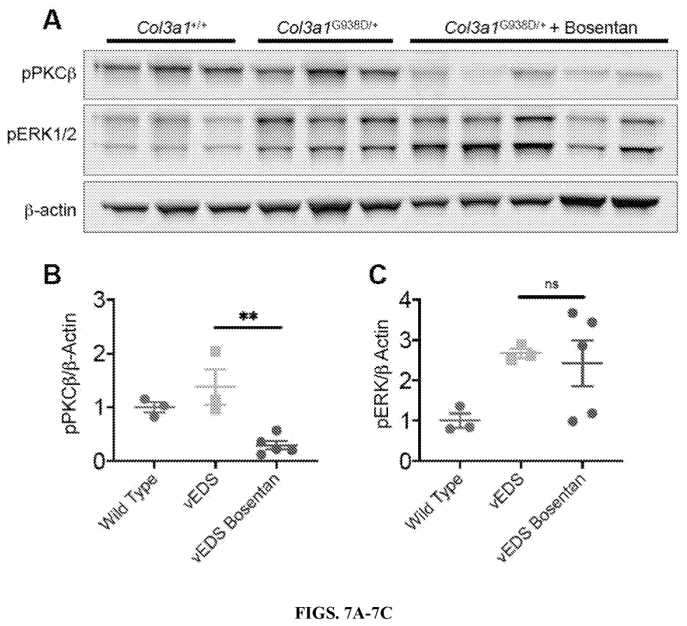

Previously the inventor demonstrated that elevated p-PKC and p-ERK1/2 are responsible for aortic rupture risk (15). The effect of inhibiting the ET1 signaling pathway on p-PKC and p-ERK1/2 levels in the proximal descending thoracic aorta of Col3a1^{G938D} mice was examined. Strong upregulation of p-PKC and p-ERK1/2 was observed in untreated Col3a1^{G938D} aortas, whereas marked downregulation of p-PKC, but interestingly not p-ERK1/2 was found in bosentan treated Col3a1^{G938D} aortas (FIGS. 7A-7C). These data provide evidence that increased p-PKC, but not p-ERK1/2, is driven by ET-1 signaling and is associated with risk of vascular rupture in Col3a1^{G938D} mice.

### Genetic Ablation of ET1 in Endothelial Cells but not Smooth Muscle Cells is Sufficient for Prevention of Dissection in Col3a1^{G938D} Mice.

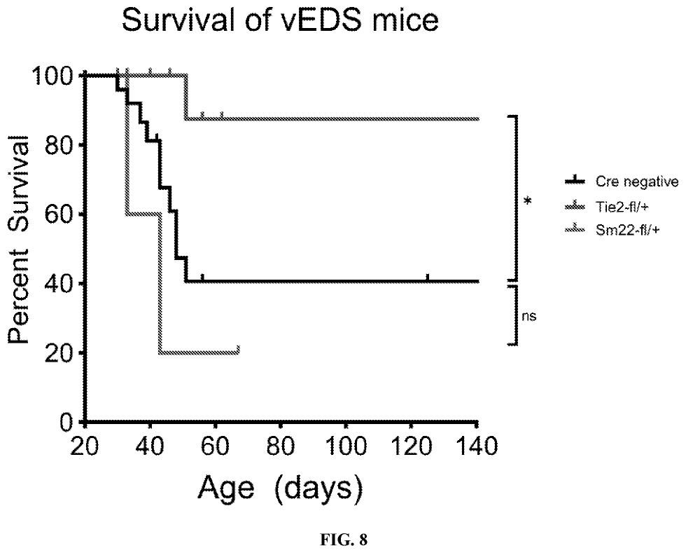

A genetic approach was utilized to examine whether ET1-mediated pathways contribute to vascular rupture risk in Col3a1^{G938D} mice. ET-1 is primarily expressed by endothelial cells, and then secreted abluminally to signal in an autocrine and paracrine manner(3, 16), however by immunostaining we detected protein expression in both the endothelial cells and vSMCs. Since ET-1 complete knock-out is perinatal lethal, a Cre-lox system was utilized to generate endothelial-cell specific and smooth muscle cell specific knock-down and knock-out of ET1. Col3a1^{G938D} mice that did not have Cre (Col3a1^{G938D};ET1^{+/+}, Col3a1^{G938D};ET1^{fl/+}, and Col3a1^{G938D};ET1^{fl/fl}) died from vascular rupture at the expected rate, with a median survival of 48 days (FIG. 8). Col3a1^{G938D} mice with a knock-down of ET1 in vSMCs (Sm22-Cre⁺; ET1^{fl/+}) also died from vascular rupture at the expected rate, with a median survival of 42 days (FIG. 8). Col3a1^{G938D} mice with a knock-down of ET1 in endothelial cells (Tie2-Cre⁺; ET1^{fl/+}) had significantly improved survival, with 87 percent of mice surviving greater than 6 months (FIG. 8). Their survival was improved over bosentan-treated mice, providing evidence that more specific or more complete ET1 antagonism is required for maximal protection. These results indicate that endothelial cell-derived ET1 contributes to vascular rupture risk in Col3a1^{G938D} mice.

### Inhibition of PKC/ERK Also Ameliorates ET1 Upregulation in Col3a1^{G938D} Aortas.

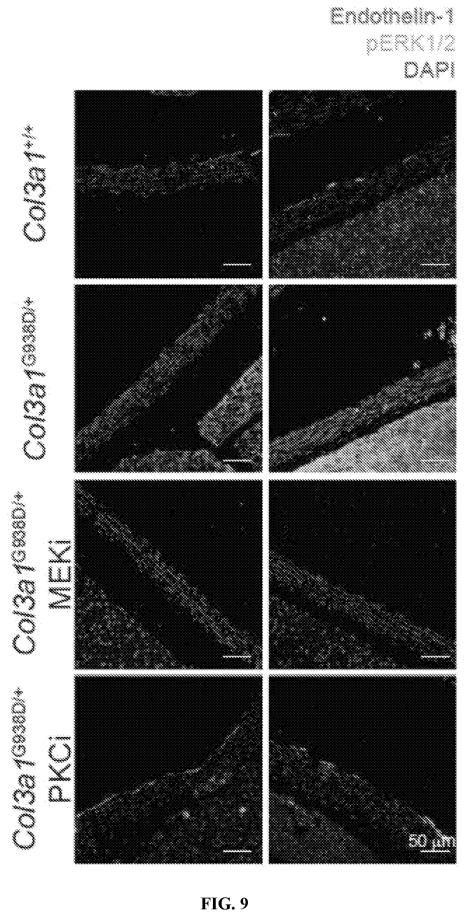

Previously it was demonstrated that elevated p-PKC and p-ERK1/2 are responsible for aortic rupture risk (15). Given that bosentan treatment decreased p-PKC but not p-ERK1/2 levels, ET1 protein levels were examined in mice treated with a PKC antagonist, ruboxistaurin, and a MEK antagonist, cobimetinib. Interestingly, increased ET1 protein levels were found to be attenuated with either MEK inhibition or PKC inhibition, providing evidence of the presence of a positive feedback loop between ET1 and the PKC/ERK axis (FIG. 9). These results indicated that type III collagen may play a role in regulation of local ET1 signaling and that EDNRA/B are candidate Gαq receptors that contributes to vascular rupture risk in vEDS, possibly through a feedback loop mechanism.

### ET1 is not Significantly Increased in the Serum or Urine of Col3a1^{G938D} Mice.

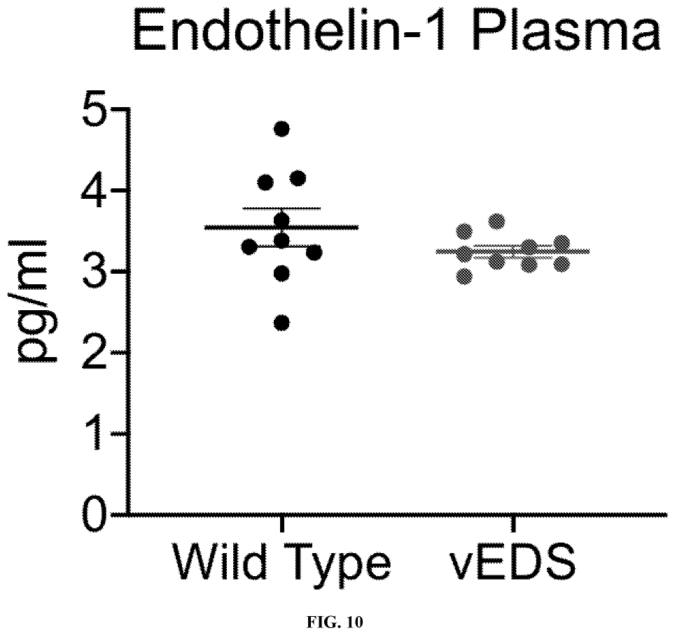

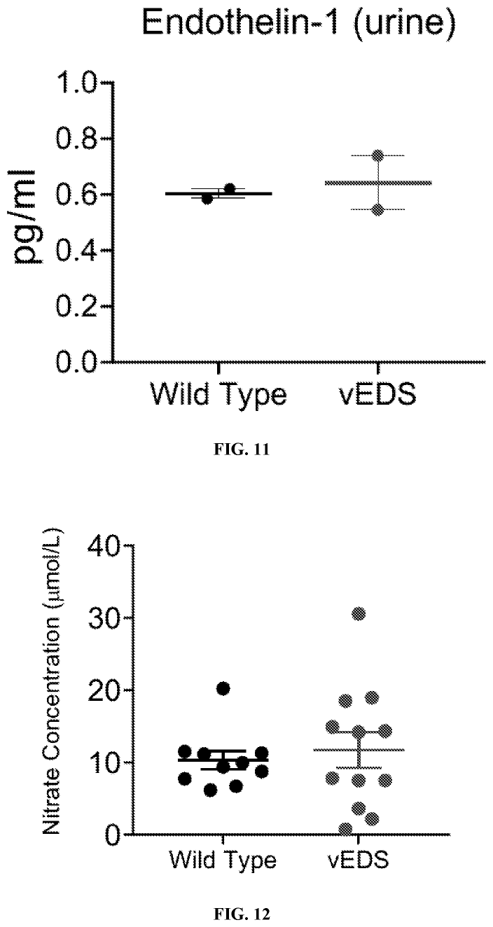

Given that ET1 is secreted by endothelial cells, it was hypothesized that differences in ET1 protein levels would be detectable in the serum of Col3a1^{G938D} mice. ET1 protein levels were also measured in the urine of Col3a1^{G938D} mice, so that the levels could be measured serially, hypothesizing that increases in ET1 would correlate with increased risk of vascular rupture. However, there was no detection of any differences in ET1 protein levels in the serum or urine of Col3a1^{G938D} mice, as measured by an ET1 ELISA (FIGS. 10 and 11). Given that ET1 is thought to be secreted abluminally, there may not be significant secretion into the blood or urine. However, differences in ET1 protein levels can be detected in other collagen vascular disorders, such as systemic sclerosis, Raynaud's phenomenon, Takayasu's Arteritis, and thromboangiitis obliterans (1, 17), and so further optimization or potentially investigation into serum or urine from humans with vEDS is warranted. Another possible biomarker for ET1 is Nitric Oxide (18, 19), however the extremely short half-life of this molecule makes it difficult to study in vivo. As a result, it's more stable byproduct, nitrate, is often measured instead. With a low number of samples, no differences were detected in nitrate concentration from serum samples of Col3a1^{G938D} mice (FIG. 12), however further optimization and investigation is warranted.

### Discussion

There are multiple mechanisms by which a collagen deficiency could lead to abnormal PLC/IP3/PKC/ERK signaling. Known physiologic activators include receptor tyrosine kinases, integrins, and G_{αq} GPCRs(11, 20-23). A deficiency of type III collagen has been associated with alterations in the level and repertoire of integrin receptors expressed at the cell surface(24, 25). Alternatively, vEDS might be associated with changes in the expression or localization of integrin ligands. While the expression profiling analyses herein, did not reveal suggestive changes in mRNA expression, these possibilities remain incompletely explored. There are no proposed mechanisms by which type III collagen deficiency would increase the expression or activity of G_{αq} GPCRs, including the angiotensin II, thrombin, and endothelin receptors. None showed enhanced expression in the vEDS descending thoracic aorta at an RNA level. Although others have shown that angiotensin II administration promotes thoracic aortic dissection in Col3a1 haploinsufficient mice (26), angiotensin II receptor blockers (ARBs) did not improve survival in Col3a1^{G938D/+} animals, suggesting that angiotensin II receptor signaling is not driving vascular rupture in our model (FIGS. 2-6). The adhesion GPCR GPR56 uses type III collagen as a ligand and is expressed in the aorta, but complete type III collagen deficiency phenocopies the polymicrogyria phenotype seen upon loss of function of GPR56, making a gain-of-function in vEDS difficult to reconcile(21, 22, 27).

By pharmacologically targeting tyrosine kinase receptors and G_{αq} GPCRs expressed within the aorta, it was observed that only EDNRA/B antagonism improved survival from vascular events and normalized PKCβ phosphorylation in vEDS mice. In addition, increased ET1 was identified, the ligand for EDNRA/B, in vEDS aortas, localized to both the intima and media of the vessel wall, and elevated ET1 levels were normalized by disease modulation through PKC and ERK inhibition. ET1, a potent vasoconstrictor released by endothelial cells, has known associations with vascular disease, and its expression is increased in vSMCs with oxidative stress (4). In vEDS aortas, elevations in ET1 were seen at the protein but not the RNA level, indicating that post-transcriptional or post-translational ET1 regulation is involved. ET1 is secreted as a proprotein and activated by endothelin converting enzyme (ECE1). ECE1 is expressed on the cell surface of vSMCs and, in disease states, increases in ECE1 activity correlate with increases in ET1 expression in the vascular media (5, 7-9), similar to what was seen by immunohistochemistry in vEDS mice. Identifying the relationship between Col3a1 and ECE1 activity may be an important to link vEDS-related mutations to dysregulated ET1 expression. That is, the relationship between Col3a1 and ECE1 activity appears to be important in the link between vEDS-related mutations and dysregulated ET1 expression.

The data also strongly indicate the presence of a positive feedback loop, as endothelin receptor antagonism decreased PKC activation, while PKC antagonism decreased ET1 levels in the vascular wall. ECE1 activity has been shown to be regulated by PKC in multiple contexts, which may contribute to the positive feedback loop seen in vEDS aortas(28-30). Taken together these results indicate that type III collagen may be required for regulation of local ET1 signaling and that there is a complex interplay between endothelial cells and vSMCs, resulting in a positive feedback loop mechanism that leads to increased vascular rupture risk.

The results herein provide evidence for a targetable signaling abnormality that contributes to the pathogenesis of vEDS and illustrate the promise of therapeutic strategies aimed at inhibition of ET1 signaling. While elevated vascular ET1 expression was not associated with increased circulating levels, the data provide evidence that circulating ET1 or byproducts of ET1 signaling may be used as biomarkers of vascular disease risk, progression, and/or therapeutic response in patients with vEDS.

### REFERENCES

1. D. Abraham, M. Dashwood, Endothelin—role in vascular disease. Rheumatology. 47, v23-v24 (2008).
2. H. Yanagisawa, R. E. Hammer, J. A. Richardson, S. C. Williams, D. E. Clouthier, M. Yanagisawa, Role of Endothelin-1/Endothelin-A receptor-mediated signaling pathway in the aortic arch patterning in mice. The Journal of Clinical Investigation. 102, 22 (1998).
3. M. Houde, L. Desbiens, P. D'Orleans-Juste, in Advances in Pharmacology (Academic Press Inc., 2016), vol. 77, pp. 143-175.
4. J. Ruef, M. Moser, W. Kübler, C. Bode, Induction of endothelin-1 expression by oxidative stress in vascular smooth muscle cells. Cardiovascular Pathology. 10, 311-315 (2001).
5. C. Ihling, T. Szombathy, B. Bohrmann, M. Brockhaus, H. E. Schaefer, B. M. Loeffler, Coexpression of endothelin-converting enzyme-1 and endothelin-1 in different stages of human atherosclerosis. Circulation. 104, 864-869 (2001).
6. C. Bowen, H. Dietz, Treatment of Vascular Ehlers-Danlos syndrome and related syndromes.
7. S. Kuruppu, A. I. Smith, Endothelin converting enzyme-1 phosphorylation and trafficking. FEBS Letters (2012), doi:10. 1016/j.febslet.2012.06.020.
8. T. D. Warner, J. A. Mitchell, P. D'Orleans-Juste, K. Ishii, U. Förstermann, F. Murad, Characterization of endothelin-converting enzyme from endothelial cells and rat brain: detection of the formation of biologically active endothelin-1 by rapid bioassay. Molecular pharmacology. 41, 399-403 (1992).
9. K. Barnes, A. J. Turner, Endothelin converting enzyme is located on α-actin filaments in smooth muscle cells. Cardiovascular Research. 42, 814-822 (1999).
10. G. Remuzzi, N. Perico, A. Benigni, New therapeutics that antagonize endothelin: Promises and frustrations. Nature Reviews Drug Discovery. 1 (2002), pp. 986-1001.
11. K. Natarajan, B. C. Berk, in Transmembrane Signaling Protocols (Humana Press, New Jersey, 2006; http://link.springer.com/10.1385/1-59745-048-0:51), pp. 51-78.
12. E. Vorkapic, E. Dugic, S. Vikingsson, J. Roy, M. I. Mäyränpää, P. Eriksson, D. W\dot{a}gsäter, Imatinib treatment attenuates growth and inflammation of angiotensin II induced abdominal aortic aneurysm. Atherosclerosis. 249, 101-109 (2016).
13. J. P. Habashi, D. P. Judge, T. M. Holm, R. D. Cohn, B. L. Loeys, T. K. Cooper, L. Myers, E. C. Klein, G. Liu, C. Calvi, M. Podowski, E. R. Neptune, M. K. Halushka, D. Bedja, K. Gabrielson, D. B. Rifkin, L. Carta, F. Ramirez, D. L. Huso, H. C. Dietz, Losartan, an AT1 Antagonist, Prevents Aortic Aneurysm in a Mouse Model of Marfan Syndrome. Science. 312, 117-121 (2006).
14. K. M. Austin, L. Covic, A. Kuliopulos, Matrix metalloproteases and PAR1 activation. Blood. 121 (2013), pp. 431-439.
15. C. J. Bowen, J. F. C. Giadrosic, Z. Burger, G. Rykiel, E. C. Davis, M. R. Helmers, K. Benke, E. G. MacFarlane, H. C. Dietz, Targetable cellular signaling events mediate vascular pathology in vascular Ehlers-Danlos syndrome. Journal of Clinical Investigation. 130, 686-698 (2020).
16. A. P. Davenport, K. A. Hyndman, N. Dhaun, C. Southan, D. E. Kohan, J. S. Pollock, D. M. Pollock, D. J. Webb, J. J. Maguire, Endothelin. Pharmacological Reviews. 68 (2016), pp. 357-418.
17. K. Yamane, Endothelin and collagen vascular disease: a review with special reference to Raynaud's phenomenon and systemic sclerosis. Internal medicine (Tokyo, Japan). 33, 579-582 (1994).
18. R. M. Rapoport, Nitric oxide inhibition of endothelin-1 release in the vasculature: In vivo relevance of in vitro findings. Hypertension. 64, 908-914 (2014).
19. S. L. Bourque, S. T. Davidge, M. A. Adams, The interaction between endothelin-1 and nitric oxide in the vasculature: New perspectives. American Journal of Physiology—Regulatory Integrative and Comparative Physiology. 300, 1288-1295 (2011).
20. D. P. Judge, N. J. Biery, D. R. Keene, J. Geubtner, L. Myers, D. L. Huso, L. Y. Sakai, H. C. Dietz, Evidence for a critical contribution of haploinsufficiency in the complex pathogenesis of Marfan syndrome. The Journal of Clinical Investigation. 114 (2004), doi:10.1172/JCI200420641.
21. R. Luo, S.-J. Jeong, A. Yang, M. Wen, D. E. Saslowsky, W. I. Lencer, D. Araç, X. Piao, Mechanism for Adhesion G Protein-Coupled Receptor GPR56-Mediated RhoA Activation Induced By Collagen III Stimulation (2014), doi:10.1371/journal.pone.0100043.
22. R. Luo, Z. Jin, Y. Deng, N. Strokes, X. Piao, Disease-Associated Mutations Prevent GPR56-Collagen III Interaction. PLoS ONE. 7, e29818 (2012).
23. L. M. Luttrell, in Transmembrane Signaling Protocols (Humana Press, New Jersey, 2006), pp. 1-50.
24. N. Zoppi, M. Ritelli, M. Colombi, Type III and v collagens modulate the expression and assembly of EDA+fibronectin in the extracellular matrix of defective Ehlers-Danlos syndrome fibroblasts. Biochimica et Biophysica Acta—General Subjects. 1820, 1576-1587 (2012).
25. N. Zoppi, R. Gardella, A. De Paepe, S. Barlati, M. Colombi, Human Fibroblasts with Mutations in COL5A1 and COL3A1 Genes Do Not Organize Collagens and Fibronectin in the Extracellular Matrix, Down-regulate α2β1 Integrin, and Recruit αvβ3 Instead of α5β1 Integrin. Journal of Biological Chemistry. 279, 18157-18168 (2004).
26. J. Faugeroux, H. Nematalla, W. Li, M. Clement, E. Robidel, M. Frank, E. Curis, H. Ait-Oufella, G. Caligiuri, A. Nicoletti, A. Hagege, E. Messas, P. Bruneval, X. Jeunemaitre, S. Bergaya, Angiotensin II promotes thoracic aortic dissections and ruptures in col3a1 haploinsufficient mice. Hypertension. 62, 203-208 (2013).
27. L. Vandervore, K. Stouffs, I. Tanyalgin, T. Vanderhasselt, F. Roelens, M. Holder-Espinasse, A. Jørgensen, M. G. Pepin, F. Petit, P. Khau Van Kien, N. Bahi-Buisson, W. Lissens, A. Gheldof, P. H. Byers, A. C. Jansen, Bi-allelic variants in COL3A1 encoding the ligand to GPR56 are associated with cobblestone-like cortical malformation, white matter changes and cerebellar cysts. Journal of medical genetics. 54, 432-440 (2017).
28. A. I. Smith, R. A. Lew, W. G. Thomas, N. Tochon-Danguy, in International Journal of Peptide Research and Therapeutics (Springer, 2006), vol. 12, pp. 291-295.
29. M. Khamaisi, R. Dahan, S. Hamed, Z. Abassi, S. N. Heyman, I. Raz, Role of protein kinase C in the expression of endothelin converting enzyme-1. Endocrinology. 150, 1440-1449 (2009).
30. S. Kuruppu, N. Tochon-Danguy, A. Ian Smith, Role of Protein Kinase C in Endothelin Converting Enzyme-1 trafficking and shedding from endothelial cells. Biochemical and Biophysical Research Communications. 398, 173-177 (2010).
31. B. Vogel, H. Siebert, U. Hofmann, S. Frantz, Determination of collagen content within picrosirius red stained paraffin-embedded tissue sections using fluorescence microscopy. Methods X. 2, 124-134 (2015).
32. N. Leng, J. A. Dawson, J. A. Thomson, V. Ruotti, A. I. Rissman, B. M. G. Smits, J. D. Haag, M. N. Gould, R. M. Stewart, C. Kendziorski, EBSeq: an empirical Bayes hierarchical model for inference in RNA-seq experiments. Bioinformatics. 29, 1035-1043 (2013).
33. A. Daugherty, D. L. Rateri, I. F. Charo, A. P. Owens, D. A. Howatt, L. A. Cassis, Angiotensin II infusion promotes ascending aortic aneurysms: attenuation by CCR2 deficiency in apoE^{-/-} mice. Clinical Science. 118, 681-689 (2010).
34. M. N. Isaacs, S. N. Anidjar, P. B. Dobrin, M. B. Eichorst, G. P. Graham, Correlation of inflammatory infiltrate with the enlargement of experimental aortic aneurysms. Journal of Vascular Surgery. 16, a35585 (1992).
35. J. P. Habashi, E. G. MacFarlane, R. Bagirzadeh, C. J. Bowen, N. N. Huso, Y. Chen, D. Bedja, T. J. Creamer, G. Rykiel, M. Manning, D. Huso, H. C. Dietz, E. M. Gallo, R. Bagirzadeh, C. J. Bowen, N. N. Huso, Y. Chen, D. Bedja, T. J. Creamer, G. Rykiel, M. Manning, D. Huso, H. C. Dietz, Oxytocin Antagonism Prevents Pregnancy-Associated Aortic Dissection in a Mouse Model of Marfan Syndrome. Science Translational Medicine. 11, eaat4822 (2019).

### Other Embodiments

From the foregoing description, it will be apparent that variations and modifications may be made to the disclosure described herein to adopt it to various usages and conditions. Such embodiments are also within the scope of the following claims.

All citations to sequences, patents and publications in this specification are herein incorporated by reference to the same extent as if each independent patent and publication was specifically and individually indicated to be incorporated by reference.

1. A method of treating a connective tissue disorder, comprising:
administering a therapeutically effective amount of an agent which modulates endothelin receptor activation, expression or function, and/or inhibits endothelin-1 (ET1) expression or binding to the endothelin receptor (EDNRA/B), thereby treating the connective tissue disorder.

2. The method of claim 1, wherein the connective tissue disorder is Ehlers-Danlos Syndrome (EDS).

3. The method of claim 2, wherein the Ehlers-Danlos Syndrome (EDS) is hypermobile EDS, classical EDS, kyphoscoliosis EDS, arthrochalasia EDS, dermatosparaxis EDS, brittle cornea syndrome, classical-like EDS, spondylodysplastic EDS, musculocontractual EDS, myopathic EDS, periodontal EDS, cardiac-valcular EDS, or vascular EDS (vEDS).

4. The method of claim 2, wherein the Ehlers-Danlos Syndrome (EDS) is vascular EDS.

5. The method of claim 1, wherein the agent comprises an antibody or fragment thereof, a polypeptide, a small molecule, a nucleic acid molecule, or any combination thereof.

6. The method of claim 1, wherein the agent inhibits endothelin-1 (ET1) expression or binding to the endothelin receptor (EDNRA/B).

7. The method of claim 1, wherein the agent is a small molecule endothelin receptor antagonist.

8. The method of claim 1, wherein the agent comprises bosentan, or a pharmaceutically acceptable salt thereof.

9. The method of claim 8, wherein the effective amount of the bosentan or the pharmaceutically acceptable salt thereof is from about 0.001 mg/kg to 250 mg/kg body weight.

10. The method of claim 1, wherein the agent comprises one or more of sitaxentan, ambrisentan, macitentan and/or tezosentan.

11. The method of claim 1, further comprising administering an agent that modulates the activity or expression of protein kinase C (PKC), mitogen-activated protein kinase (MEK) or the combination thereof.

12. The method of claim 11, wherein a modulator of PKC activity or function is administered and comprises ruboxistaurin or pharmaceutically acceptable salts thereof.

13. The method of claim 11, wherein a modulator of MEK activity or function in administered and comprises trametinib, binimetinib, selumetinib, cobimetinib or pharmaceutically acceptable salts thereof.

14. The method of claim 1, further comprising administering ruboxistaurin, cobimetinib pharmaceutically acceptable salts thereof or the combination thereof.

15. The method of claim 1, further comprising administering a modulator of type III collagen expression or function.

16. The method of claim 1, wherein detection of ET1 signaling and/or levels of ET1, nitric oxide, nitrate, or nitrite in patient sample are indicative of vascular disease risk, progression, and/or therapeutic response in the patient.

17. A method of treating a vascular Ehlers-Danlos Syndrome in a subject in need thereof, the method comprising: administering to the subject an effective amount of bosentan or a pharmaceutically acceptable salt thereof.

18. The method of claim 17, wherein the effective amount of the bosentan or the pharmaceutically acceptable salt thereof is from about 0.001 mg/kg to 250 mg/kg body weight.

19. The method of claim 17, further comprising administering an agent for modulating expression or activity of a COL3A1 gene, correcting mutations of a COL3A1 gene or the combination thereof.

20. The method of claim 19, wherein the agent is a gene editing agent.

21. The method of claim 17, wherein detection of ET1 signaling and/or levels of ET1, nitric oxide, nitrate, or nitrite in patient sample are indicative of vascular disease risk, progression, and/or therapeutic response in the patient.

22. A method of treating a vascular Ehlers-Danlos Syndrome in a subject in need thereof, the method comprising: administering to the subject an effective amount of a small molecule endothelin receptor antagonist.

23. A method of treating a vascular Ehlers-Danlos Syndrome in a subject in need thereof, the method comprising: administering to the subject an effective amount of sitaxentan, ambrisentan, macitentan and/or tezosentan.

24-25. (canceled)
